(19) United States
(12) Patent Application Publication
(10) Pub. No.: US 2025/0270624 A1
(43) Pub. Date: Aug. 28, 2025

(54) PCR MULTIPLEXING THROUGH TARGET SEQUENCE-INDEPENDENT REPORTER MOLECULES WITH DISTINGUISHABLE SIGNAL STRENGTHS

(71) Applicant: HAHN-SCHICKARD-GESELLSCHAFT FÜR ANGEWANDTE FORSCHUNG E. V., Stuttgart (DE)

(72) Inventors: Michael LEHNERT, Stuttgart (DE); Silvia CALABRESE, Stuttgart (DE); Anja MARKL, Stuttgart (DE); Franziska SCHLENKER, Stuttgart (DE); Elena KIPF, Stuttgart (DE)

(21) Appl. No.: 18/858,312

(22) PCT Filed: Apr. 21, 2023
(86) PCT No.: PCT/EP2023/060507

§ 371 Date: Oct. 19, 2024

(30) Foreign Application Priority Data
Apr. 22, 2022 (EP) 22169459.9
Dec. 9, 2022 (EP) 22212538.7

(51) Int. Cl. C12Q 1/6818; C12Q 1/682

(52) U.S. Cl. CPC C12Q 1/6818; C12Q 1/682

(57) ABSTRACT

The invention relates to a method for specific detection of at least two nucleic acid target sequences through at least two target sequence-independent reporter molecules in the same detection channel, wherein at least two mediator probes, comprising at least one probe sequence and one mediator sequence, and at least two target sequence-independent reporter molecules of a first and second type, each comprising at least one label with a respective signal intensity maximum in the same detection channel, are employed and wherein the at least two nucleic acid target sequences are detected by means of a nucleic acid detection reaction, due to a signal intensity and/or emission spectrum that is characteristic therefor. The invention also relates to a kit for performing the method.

### TECHNICAL BACKGROUND

Precise and sensitive nucleic acid detection methods, as a further development of real-time PCR, in particular digital PCR, have revolutionized molecular diagnostics in recent years, as they are superior to standard PCR in the detection of rare gene targets (Garcia-Murillas et al. 2015; Milbury et al. 2014). In digital PCR (dPCR), the reaction mix and the nucleic acids contained therein (e.g. ctDNA or DNA) are divided into thousands of reaction spaces, whereby the target sequences are separated from each other and their amplification and detection are facilitated, especially with respect to rare target sequences. A common method here is the microfluidic dripping of the reaction mix with the nucleic acid to be detected in oil or division into fixed microcavities, whereby each unit formed forms a closed reaction space for amplification and detection, for example during a PCR. If a nucleic acid target sequence is present in a droplet, it is amplified by PCR (or other amplification methods). The individual amplificates are usually detected by sequence-specific fluorogenic nucleic acid probes, whereby corresponding fluorescence signals are generated. In addition to absolute quantification, dPCR also has the advantage of reducing the influence of inhibitors. Since quantification in digital PCR is performed as an endpoint analysis, a “signal cluster” (also referred to as a population) in the data space can be assigned to a specific DNA or cDNA target sequence via the fluorescence signal strength, which enables an alternative type of data classification (Quan et al. 2018; Whale et al. 2016).

One relevant aspect of dPCR is multiplexing, as different nucleic acid target sequences often occur in very low concentrations in samples and it is highly relevant to detect as many target sequences as possible within a sample. The most common multiplex methods in dPCR are based on the fact that DNA reporter molecules generate a fluorescence signal for each DNA target sequence in a spectral range that corresponds to the detection range of a detection channel. One DNA target sequence can therefore be detected per detection channel on the device side. However, the degree of multiplexing of a device is initially limited to the number of available detection channels. The dPCR devices currently available on the market have between 2-6 different fluorescence detection channels, which means that a maximum of 6 target sequences can be analyzed on the device side. A major problem here is that wavelengths can overlap with a high number of detection channels, resulting in a lower specificity and sensitivity due to the resulting crosstalk.

One method for increasing the degree of multiplexing uses combinatorial approaches, e.g. by combining Taqman probes and DropOff probes as a target sequence of specific reporter molecules. In this case, the combinatorics of the signals generated by these probes also results in a higher degree of multiplexing (Madic et al. 2018; Stilla Technologies; Corné et al. 2021). As the individual mutations are only detected indirectly in some cases, these methods are also very complex in their development and very demanding in their evaluation, and certification or transfer to new target panels is correspondingly complex.

In addition, there are other methods for increasing the degree of multiplexing in digital PCR, which aim to generate additional populations in the data space. A US patent from Bio-Rad (U.S. Pat. No. 9,921,154B2) describes a multiplex digital PCR analysis of more target sequences than there are optical channels available, taking into account other sample-specific aspects. However, the patent does not disclose any new assay features or methods for generating such target sequence populations. According to the current state of knowledge, these methods can only be used in cases where there is per se a clear multiple occupancy of droplets with different DNA target sequences or in cases where signals are generated by target sequence-specific reporter molecules such as Taqman probes or non-sequence-specific intercalating dyes, which in themselves, however, do not represent an innovation over the prior art. It is described that different target sequence-specific probes can use e.g. different fluorophores as labels for this purpose or can carry multiple fluorophore labels to generate multiple populations in the same detection channel. Since the signals of the probes described here are suppressed in the ground state by FRET quenching with low efficiency, the initial signal suppression is comparatively low, which is why effective modeling of the signal strength and thus separation of the populations in the data space is only possible to a limited extent. Since these labeled probes are also target sequence-specific, extensive optimization of their fluorescence properties is very inefficient, as new labeled probes have to be synthesized each time and this procedure has to be restarted when transferring to other sequences. Similarly, the multiple modification of such target sequence-specific probes with fluorophores is very complex and also requires modification with additional quenchers to suppress the signal sufficiently in the ground state to avoid unspecific signal populations in the data space. Such multiple modifications are very complex to synthesize and usually require extensive optimization, which is why they are rarely used for target sequence-specific reporter molecules, as this procedure is very inefficient.

The most common variant in this intensity multiplexing is therefore to vary the concentration of the different types of target sequence-specific reporter molecules (e.g. Taqman probes) while maintaining uniform and simple fluorescent labels per detection channel (FIG. 1 right) (Pecoraro et al. 2016; Whale et al. 2016). This can create additional populations in the data space of a graph. Due to the use of target sequence-specific fluorogenic reporter molecules, signal generation is directly dependent on the target sequence or the PCR system. Accordingly, this direct detection poses the problem of a high optimization effort, as the strong variance (data points that lie between different populations) leads to blurred signal populations that reduce precision (intensity multiplexing). The individual PCR components and in particular the fluorogenic reporters with identical fluorescent labels must be well matched to each other, e.g. in orthogonal concentration ratios, which is not always possible in practice. This method can also be supplemented by combining it with other dyes that exhibit additional fluorescent labels, which have their emission maximum between different fluorescence detection channels and can therefore be detected equally in multiple fluorescence channels. This results in corresponding populations between the other clusters in the data space, which becomes increasingly abstract with increasing dimensions of the data space due to additional detection channels. This makes the development of corresponding multiplex detections and, in particular, their approval very complex. A further variation of intensity multiplexing uses even more complex concentration variations to offset the populations in the data space and thus make them less susceptible to variance (Hughesman et al. 2016).

A detection method for nucleic acids which, in contrast, does not use target sequence-specific reporter molecules and is based on a separation of nucleic acid detection by target sequence-specific mediator probes and signal generation by target sequence-unspecific universal reporters is mediator probe PCR, which was patented by the University of Freiburg in 2012 (WO2013079307A1, Faltin et al. 2012). This method is designed for colorimetric multiplexing with standardizable reporter molecules. A signal is recorded for each detection channel, which can only be assigned to one activated universal reporter in a homogeneous reaction, regardless of its signal strength. It has already been observed that different fluorophores can lead to different signal intensities and signal curves under real-time PCR conditions and that the performance characteristics of the corresponding multiplex real-time PCRs can be optimized by maximizing the signal generation of the target sequence-unspecific fluorogenic universal reporter molecules (Lehnert et al. 2018; Kipf et al. 2022). However, modeling the signal strengths in real-time PCR has no practical added value, as the signal curves in the same detection channel cannot be distinguished using this approach. In the colorimetric digital multiplex Mediator Probe PCR, it was also shown that maximizing the fluorescence signal generation leads to a better separation of the populations in the data space compared to the negative controls (Schlenker et al. 2021b).

During the same period, a technology was developed by Seegene Inc. which is also based on the separation of DNA sequence detection and signal generation via two detection molecules in realtime PCR. Here, an unlabeled PTO probe binds to a DNA target sequence, is cleaved and releases a fragment, which then forms an extended duplex with a fluorescently labeled target sequence-unspecific detection molecule (CTO molecule). This process is used to influence the signal generation over different lengths of these detection molecules. This makes it possible, for example, to differentiate between multiple target sequences in the same detection channel by reading out the signal at defined, predetermined temperatures. Another patent also uses the readout at different temperatures in real-time PCR in one approach in the same detection channel and can therefore be interpreted as a further development of the Seegene technology mentioned above (PCT/CN2018/084794). Here, too, an extended reporter molecule is melted after detection. In contrast to the previous patent, a reporter molecule can be used to differentiate between different target sequences. As temperature control during readout cannot currently be implemented as standard in dPCR in commercially available devices, it is not possible to transfer this directly to dPCR.

Hahn-Schickard had already worked on increasing the degree of multiplexing in PCR by generating virtual fluorescence channels via photobleaching (WO2016087637A1) (Schuler et al. 2016). In order to combine this approach with the advantages of separating signal generation and detection in digital mediator probe PCR, a step was added to a digital mediator probe PCR using various universal reporter types with different fluorophore labels, in which these were bleached by light from an LED lamp (bleaching) and read out in the same fluorescence detection channel. Depending on the selected fluorophore, a decrease in signal intensities can be observed as a result of bleaching, whereby the populations in the data space of a diagram can be separated and additional, so-called virtual channels, are generated (Schlenker et al. 2021a). However, as bleaching technology requires additional devices and process steps that cannot currently be supplied with commercially available devices, implementation in existing dPCR platforms is not possible due to technical and regulatory hurdles. In this approach, however, the fluorophores selected as labels for the universal reporters did not yet lead to different fluorescence signal strengths before the bleaching step, which enabled a uniform separation of the populations in the data space of a diagram if more than one target sequence was included per reaction. The universal reporters used did not have the necessary configurations of fluorophores and quenchers to enable monochrome identification. Due to the high scattering of the fluorescence signal intensities of the reporter labels, no clear distinction could be made between a single fluorescence signal population with higher “variance” and a second signal population in the data space. In particular, this technology does not allow an obvious evaluation of whether a multiplex reaction is one or two populations, and thus does not provide the possibility of direct multiplexing in the same detection channel by target sequence-unspecific reporters (Schlenker et al. 2021a).

### OBJECTIVE OF THE INVENTION

Approaches for adjusting the signal strengths on target sequence-independent reporter molecules (generated by e.g. different fluorophores and/or quenchers) in such a way that in one reaction a plurality of distinguishable signal clusters are generated in the same detection channel or in the corresponding area of the multidimensional data space and thereby the advantages of indirect signal generation in terms of assay precision and robustness are exploited in order to circumvent the previously described problem of colorimetric multiplexing or target sequence-specific intensity multiplexing in a digital PCR are still missing in the prior art. So far, it has only been described to multiplex unspecific reporter molecules monochromatically in a digital amplification by target sequence and thereby achieve higher degrees of multiplexing by introducing additional process steps (e.g. bleaching). This complicates the process or the evaluation, reduces the precision, leads to additional optimization effort and complicates, for example, approval procedures. It is therefore the objective of the present invention to increase the number of detectable nucleic acid target sequences in a detection reaction beyond the number of detection channels on the device side.

### SUMMARY OF THE INVENTION

The present invention solves this objective by the method of the dependent and independent claims.

Therefore, in one aspect, the invention relates to a method for the specific detection of at least two nucleic acid target sequences by at least two target sequence-independent reporter molecules in the same detection channel, comprising the steps of:
a. Providing at least a first and a second nucleic acid target sequence,
b. Providing at least a first and a second mediator probe, each comprising an oligonucleotide,
   wherein the oligonucleotide of a first mediator probe comprises a mediator sequence and a probe sequence, wherein the mediator sequence has an affinity for a target sequence-independent reporter molecule of a first type and the probe sequence exhibits an affinity for a first nucleic acid target sequence,
   wherein the oligonucleotide of a second mediator probe comprises a mediator sequence and a probe sequence, wherein the mediator sequence has an affinity for a target sequence-independent reporter molecule of a second type and the probe sequence exhibits an affinity for a second nucleic acid target sequence,
   wherein the at least first and second mediator probes have no labels, or preferably no signal-generating labels,
C. Providing at least two target sequence-independent reporter molecules of at least a first and a second type, each comprising at least one label with a measurable signal in the same detection channel, and a nucleic acid sequence which has a specific affinity for at least one mediator sequence,
   wherein each type of the at least two target sequence-independent reporter molecules is characterized by the respective at least one label in such a way that it has a signal intensity which is preferably unique for the respective type of the target sequence-independent reporter molecules and is distinguishable from the signal intensities of the labels of all other target sequence-independent reporter molecule types and enables direct assignment to the respective nucleic acid target sequence,
d. Performing a nucleic acid detection reaction, wherein at least one mediator sequence of at least a first mediator probe is released when its probe sequence binds to at least a first nucleic acid target sequence,
   wherein the at least one released mediator sequence binds to the at least one target sequence-independent reporter molecule of a first type, wherein a signal is generated by the at least one label which is characteristic by its signal intensity and/or emission spectrum for the first nucleic acid target sequence assigned to the respective target sequence-independent reporter to which the at least one probe sequence of the at least one first mediator probe has bound,
   and optionally wherein at least one mediator sequence of at least one second mediator probe is released when its probe sequence binds to at least one second nucleic acid target sequence, wherein the at least one released mediator sequence binds to the at least one target sequence-independent reporter molecule of a second type, wherein the at least one label generates a signal, whose signal intensity and/or emission spectrum is characteristic for the second nucleic acid target sequence assigned to the respective target sequence-independent reporter, to which the at least one probe sequence of the at least one second mediator probe has bound,
e. Detecting the signal(s) generated under step d., comprising detecting the signal(s) in a detection channel and/or analyzing the signal(s), the signal strength(s) and/or the emission spectrum(s) of the signal(s) and/or the thus generated cluster(s) of the signal(s) in a data space, preferably wherein the signals of different target sequence-unspecific reporter molecules can also be distinguished by their signal strengths in identical detection channels or the corresponding regions of a data space.

In embodiments, a first mediator probe thus comprises an oligonucleotide, which itself comprises a mediator sequence, and a probe sequence. Thus, in embodiments, a second mediator probe further comprises an oligonucleotide, which itself comprises a mediator sequence, and a probe sequence. Preferably, the mediator sequence of the first mediator probe differs from the mediator sequence of the second mediator probe. Preferably, the probe sequence of the first mediator probe differs from the probe sequence of the second mediator probe.

In other words, in embodiments of the method according to the invention, the mediator sequence of the first mediator probe differs from the mediator sequence of the second mediator probe, and the probe sequence of the first mediator probe differs from the probe sequence of the second mediator probe, respectively. Preferably, therefore, the probe sequence of the second mediator probe also has an affinity for a “second” nucleic acid target sequence, which differs from the “first” nucleic acid target sequence (e.g. in its nucleic acid sequence), to which in turn the probe sequence of the first mediator probe has an affinity. The same also applies to optionally present and optionally used further (third, fourth, etc.) mediator probes.

In embodiments, step c. comprises providing at least two target sequence-independent reporter molecules of at least a first and a second type, each comprising at least one label having a respective measurable signal in the same detection channel and/or a respective signal intensity maximum in the same detection channel, and a nucleic acid sequence each having a specific affinity for at least one mediator sequence.

According to the invention, a “first” nucleic acid target sequence and a “second” nucleic acid target sequence preferably differ in their nucleic acid sequence and/or their epigenetic modifications. The same also applies to optionally present and optionally detectable further (third, fourth, etc.) nucleic acid target sequences.

In preferred embodiments of the method according to the invention, target sequence-independent reporters in the form of labeled oligonucleotides are assigned to nucleic acid target sequences. These are activated by the release of a further oligonucleotide as part of the nucleic acid detection reaction, thereby generating a signal. Different types of reporter molecules are each modified with different labels (e.g. fluorophores and/or quenchers and/or fluorophores and quenchers in different numbers) and have different DNA sequences, which indirectly enable assignment to a target sequence. In identical detection channels, different target sequence-independent reporter molecule types generate signals of different signal strengths due to their different labels. As a result, the signals of different reporter molecule types, each with different labels, can also be distinguished in the same detection channel or the corresponding spanned data space (spanned by the axes in a multidimensional representation or by the different detection channels in a purely mathematical comparison/analysis and/or an algorithmic analysis) on the basis of the different signal intensities and target sequences can be assigned. According to the invention, the signals of at least two different reporter complexes can preferably be detected in the same detection channel or the corresponding area of a data space. The fact that these reporter molecules are independent of the target sequence makes it possible for the first time to develop reporter molecules with particularly well distinguishable signal strengths within the same detection channel. As these are target sequence-independent, they can also have suitable label configurations that enable efficient signal modeling. For example, their signals can be suppressed in the initial state by contact quenching, whereby they show greater differences in signal generation in the activated state or have multiple labels with fluorophores and/or quenchers. This allows users to increase the number of target sequences detectable in a reaction beyond the number of detection channels without the need to purchase additional equipment, expand equipment or implement further complex process steps, which represents enormous added value and ensures high cost efficiency. Existing laboratory processes can be retained and already certified devices can continue to be used without the need for re-implementation and, if necessary, re-certification. Furthermore, this enables efficient optimization of these target sequence-independent reporter molecules, as they can subsequently be used for further target sequences and do not have to be developed and optimized from scratch in target sequence dependence. Thus, the invention achieves a simplified and efficient process optimization for the standard detection of different target sequences and a reduction of the required work and costs.

The method according to the invention enables precise efficient (direct) monochromatic multiplexing by target sequence-unspecific reporter molecules. It thereby enables the efficient target sequence-independent optimization of the reporter molecules and thus improves the separation of the signal populations in the same detection channel or the corresponding area of the data space. This enables precise detection of additional nucleic acid target sequences beyond the number of detection channels of the device. This is possible because the use of target sequence-independent reporter molecules significantly simplifies and improves symmetrical multiple labeling with fluorophores and quenchers and/or labeling with different fluorophore labels for the same detection channel in a suitable configuration and/or modeling of fluorescence signal generation, e.g. by contact quenching, compared to target sequence-specific reporter molecules.

This is done using target sequence-independent reporter molecules (e.g. universal reporters), which are labeled oligonucleotides that can each be (indirectly) assigned to a nucleic acid target sequence to be detected and which are activated by the binding of a released further oligonucleotide as part of a nucleic acid detection reaction (e.g. PCR or digital PCR). To differentiate the signals representing/indicating the presence of different nucleic acid target sequences in the same detection channel or in the corresponding area of the data space (the graphical representation of the detected signals), at least two target sequences to be detected are each assigned to two different types of target sequence-unspecific reporter molecules, which generate a signal with different signal strength in the same detection channel.

An advantage of the method according to the invention is the possibility to simultaneously detect a plurality of target nucleic acid sequences in the same detection channel of a (PCR) device, e.g. the channel for the detection of a red fluorescence signal. This simultaneous detection in a single detection channel is achieved by using different target sequence unspecific reporter molecules, each comprising labels which generate different signals with similar fluorescence emission spectra and which can be detected simultaneously in the same fluorescence detection channel of a device, e.g. a digital PCR device, and still be distinguishable from each other. With this feature, the invention enables monochrome multiplexing, namely the detection of a plurality of targets within a single (“monochrome”) fluorescence detection channel (with a specific wavelength range).

In some digital PCR methods of the prior art, a first or a second target sequence is detected in emulsion droplets, each isolated in one droplet, by using two labeled probes, each of which generates a homogeneous signal that can be distinguished from unspecific amplifications. In the method according to the invention, no labeled probes are used, which has the advantage that the labeled reporter molecules can be optimized to a greater extent than would be possible with the known methods of the prior art, since the reporter molecules according to the invention can subsequently be used for further assays. In addition, the reporter molecules according to the invention can preferably adopt only one configuration in which contact quenching occurs. This preferred property preferably enables or supports (direct) monochromatic multiplexing in the form according to the invention.

A further advantage of the method according to the invention is that the desired detection properties can be precisely adjusted by selecting the respective reporter molecules used, which are independent of the target sequence to be detected. The connecting piece between reporter molecules with desired properties (e.g. fluorescence intensity and/or color), which is easy to produce and design, are the mediator probes, which comprise a probe sequence and a mediator sequence. For the individualization of the target nucleic acid to be detected, only the two different sequence components of the mediator probe need to be provided. These can then be combined with the target sequence-unspecific reporter molecule that matches the mediator sequence. This independence allows a desired specific contact quenching to be set, enabling the detection of a plurality of distinguishable reporter molecule signals in the same detection channel. Furthermore, the optimization of the signals can be performed once independently of numerous target sequences. In addition, the method according to the invention is resource-saving, since reporter molecule sets can be reused for any number of nucleic acid target sequences, making the initial one-time development of the combinable reporter molecule signals very labor and cost efficient.

In addition, the method according to the invention enables direct detection of a nucleic acid target sequence as this can be directly assigned to a data population (signal population in the evaluation/analysis) and does not require combinatorics.

One possibility to generate different signal strengths by different labeling on reporter molecules results, for example, from the physical molecular properties of the labels, which are amplified by contact quenching after activation of the target sequence-unspecific reporter molecule:

In embodiments, therefore, the at least one label of a target sequence-independent reporter molecule is at least one fluorophore and/or at least one quencher.

Another essential property of these target sequence-unspecific reporter molecules in the efficient generation of precise distinguishable signal populations is that a labeling configuration is selected which enables signals to be generated in such a way that their signal strengths are sufficiently robust, stable and thus precise enough to remain distinguishable in the readout process without further process steps (such as bleaching). It was shown that this is possible, for example, by using configurations corresponding to the basic structure of a universal reporter or corresponding modifications in a standardized manner. Target sequence-unspecific reporter molecules can be modeled in a suitable way, whereby different signal strengths can be precisely adjusted in the detection channel (Lehnert et al. 2018).

Consequently, in embodiments, different target sequence-independent reporter molecule types differ in the signal intensity and/or emission spectrum of their at least one label.

In embodiments, the at least one label of a target sequence-independent reporter molecule comprises at least two fluorophores and/or two quenchers having the same or different emission spectrum and/or the same or different signal intensity.

In some embodiments, a reporter molecule of a first type differs from that of at least a second type by the number of labels or number of fluorophores and/or quenchers. Thus, different types of target sequence-independent reporter molecules can be distinguished by the signal strength and/or intensity of the fluorescence released upon their activation. For this purpose, different types of target sequence-independent reporter molecules may comprise different numbers and/or different fluorophores (with different emission spectra/colors and/or intensities) and/or quenchers or any combination thereof.

In preferred embodiments in which the label comprises at least one fluorophore, its fluorescence signal is preferably suppressed by contact quenching until the reporter molecule is activated. Thus, preferably no signal is released by a non-activated reporter molecule. Activation preferably takes place by the binding of a mediator sequence to the corresponding target sequence-independent reporter molecule and/or by a subsequent extension of a mediator sequence already bound to the target sequence-independent reporter molecule by a polymerase during a nucleic acid detection reaction.

Thus, in embodiments, the at least one label of a target sequence-independent reporter molecule comprises at least one fluorophore and at least one quencher, wherein contact quenching preferably occurs between the at least one fluorophore and the at least one quencher as long as no mediator sequence binds to the corresponding target sequence-independent reporter molecule, or as long as a bound mediator sequence has not been extended during the nucleic acid detection reaction.

This enables, for example, the detection of additional DNA target sequences in a suitable device as part of digital DNA amplification and fluorescence-based detection, without the need for further technical aids or varying the concentrations of reporter molecules with fluorescent labels. This allows, for example, results similar to those generated by digital mediator probe PCR (MP-PCR) in combination with photobleaching to be generated without the need for a bleaching process (e.g. via an LED or thermally), which significantly reduces the experimental effort, simplifies it and makes it possible to implement it in existing devices. Thus, the method according to the invention represents an enormously high added value compared to digital mediator probe PCR (MP-PCR) or other prior art methods, as it is directly compatible with existing platforms and can significantly simplify a possible diagnostic approval of a specific detection method. The method according to the invention offers additional new degrees of freedom in assay development, wherein the synthesis processes for corresponding target sequence-specific probes are not necessarily complicated. In addition, the sensitivity of an assay according to the invention can be significantly increased, since additional nucleic acid sequences can be included in each reaction. Another advantage is that a suitable set of target sequence-unspecific reporter molecules always generates populations in similar areas of the data space, whereby conclusions can be drawn about the activated reporter independently of the target sequences within the detection reaction and thus the same set can be used with different nucleic acid target sequence panels without time-consuming optimization.

In embodiments of the method according to the invention, the analysis of the signal, the signal strength and/or the emission spectrum of the signal under point e. comprises displaying and/or analyzing the detected signal as a function of the signal strength and/or the detection channel and/or the emission spectrum in a data space spanned by the evaluated detection channels.

In the context of the present invention, a data space is preferably generated by a plot, a diagram or a graph of the different detection channels or by a purely mathematical and/or algorithm- and/or computer-based evaluation, comparison or juxtaposition of the different detection channels, wherein the respective fluorescence signals of the target sequence-unspecific reporters used generate data points in this 1-n dimensional data space (depending on the number of detection channels required for the evaluation), which can be grouped into clusters (groups, collections).

In the context of the present invention, a data space is preferably the space which is spanned in a plot, diagram or graph by an X-axis and a Y-axis (two-dimensional plot), and optionally in the case of a three-dimensional graph additionally by a Z-axis, or in the case of a correspondingly higher-dimensional space by a further axis, and in which the data to be displayed (of the detected signals) are represented. In embodiments in which the detection channels used in the process are displayed on the axes of a graph, for example the signals detected in the red detection channel on the X-axis and the signals detected in the green detection channel on the Y-axis, the data space can also be referred to as “spanned by the detection channels”. Preferably, it is possible to clearly assign the detected signals displayed in the data space, which can preferably be displayed in the form of distinct signal populations, to a target sequence. This is preferably also possible if a plurality of signals specific to different target sequences have been detected in the same detection channel.

In embodiments, the reporter molecules used simultaneously in a PCR reaction differ from each other either by different types (e.g. colors, emission spectra, and/or intensities) of fluorophores and/or different types of quenchers and/or by the number of labels on the different reporter molecules. In embodiments of the monochromatic multiplexing according to the invention, at least two target nucleic acids in the same detection channel are indirectly detected by reporter molecules with different or distinguishable signals. In embodiments in which more than two target nucleic acids are to be detected, the signals to be generated for the detection of the target nucleic acids can, if necessary, be divided between a plurality of detection channels (and the labels of the different reporter molecule types can be selected accordingly), wherein preferably at least two target nucleic acids in the same detection channel are detected indirectly by reporter molecules with different or distinguishable signals.

In embodiments, at least a first and a second nucleic acid target sequence, or even at least a first, a second and a third, or even a fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or even more target sequences can be detected or their presence can be detected.

According to the number of nucleic acid target sequences to be detected, at least a first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or even more mediator probes, each comprising an oligonucleotide, each comprising a mediator sequence and a probe sequence, are provided.

Accordingly, depending on the number of nucleic acid target sequences to be detected, target sequence-independent reporter molecules of a first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first or even more, type are provided and used in the method or kit according to the invention as described herein.

A person skilled in the art will understand how to combine types of target sequence-independent reporter molecules and their labels according to the invention to achieve detection of desired target sequences according to the invention.

In embodiments, the labeling of the at least one target sequence-independent reporter molecule comprises at least two complementary opposing nucleobases each having at least one label or at least two opposing bases offset by one base position from a complementary base pairing each having at least one label.

The present invention enables the simultaneous detection of a plurality of target nucleic acids (multiplexing), wherein the number of simultaneously detected target nucleic acids can be greater than the number of detection channels available in the detection device. This is made possible by the fact that the reporter molecules used in each case comprise labels which, despite simultaneous detection of the signals of a plurality of different reporter molecules in the same detection channel, generate signals that can be distinguished from each other and can be represented in the analysis as distinct (non-overlapping) signal populations in two- or three-dimensional data space. In embodiments, this enables differentiation of signals indicating the presence of different target nucleic acids despite detection in the same detection channel, and, in specific embodiments, also differentiation from background noise.

In embodiments of the method according to the invention, in step d.-e. n different nucleic acid target sequences are indirectly detected by n different target sequence-independent reporter molecule types, wherein the detection of the signal generated in step d. takes place in k detection channels,
where n>k and n≥2, and
wherein at least two different target sequence-independent reporter molecule types are detected in the same detection channel in step e. and/or represented in the same region of a data space in the representation under point e.

By using the method according to the invention, it is possible in embodiments to perform data classification in the higher-dimensional data space (>3 dimensions, with more than three detection channels). In embodiments, the method according to the invention allows the number of distinguishable target sequences in a digital PCR to be at least doubled compared to the available detection channels, provided that this is not prevented per se by technical limitations on the device side, such as possible crosstalk (mutual interference of fluorescence signals; erroneous association of a fluorescence signal with the signal of another fluorophore due to the erroneous detection of fluorescence from a neighboring detection channel) between different detection channels.

In embodiments, a signal of a label of a reporter molecule is suppressed, deleted or quenched until a mediator sequence binds to the reporter molecule and is preferably extended by a polymerase as part of a nucleic acid amplification reaction. In embodiments, a spatial separation, e.g. of at least one fluorophore from at least one quencher, is achieved by the polymerase so that a signal is generated and can be detected.

In embodiments of the method according to the invention, the signal of the labeling of a target sequence-independent reporter molecule is generated by cleavage and/or separation of the target sequence-independent reporter molecule and/or by spatial separation of the at least one fluorophore and the at least one quencher.

In embodiments, the at least one target sequence-independent reporter molecule is an oligonucleotide or an oligonucleotide complex.

In embodiments of the method according to the invention, the nucleic acid detection reaction in step d. comprises an amplification procedure for DNA and/or cDNA.

In embodiments of the method according to the invention, the nucleic acid detection reaction under step d. is a PCR, RT-PCR, RPA or LAMP, wherein, in the course of DNA amplification, a mediator sequence of a mediator probe bound to a target nucleic acid is released by an enzymatic activity of a biomolecule, which then binds to a target sequence-independent reporter molecule such that a signal is generated.

In embodiments, the detection under step e. is performed as part of a digital amplification and/or signal generation.

In embodiments of the method according to the invention, the target sequence-independent reporter molecules are universal reporters and/or modular reporter complexes and the at least one (respectively) released mediator sequence is a component of a Mediator Probe PCR or Mediator Displacement LAMP.

In embodiments, the nucleic acid target sequence is derived from a conversion of another biomolecule into DNA sequence information.

In some embodiments, the presence and/or amount of an RNA sequence (an embodiment of a biomolecule) in a sample can be determined by transcribing the RNA sequence into cDNA by a reverse transcription reaction. In embodiments, this cDNA can be amplified as part of the method according to the invention and indirectly detected by a target sequence-independent reporter molecule.

In embodiments of the method according to the invention, the at least one label comprises a label selected from an electrochemically active and/or a magnetic label.

In embodiments of the method according to the invention, the signal is read out at different temperatures.

In some preferred embodiments, a mediator probe comprises an oligonucleotide and a sequence-specific probe portion that binds to the target sequence and is protected at the 3′ end. This protection at the 3′ end may be a blocking group (protecting group), e.g., a chemical block or protecting group, which in some embodiments comprises a chain of three carbon atoms. Protection of the mediator probe at the 3′ end preferably prevents (unspecific) extension of the sequence strand by a polymerase during an amplification reaction. In accordance with the invention, in embodiments, the mediator probe may comprise any protecting or blocking group suitable for preventing (unspecific) extension of the mediator probe sequence strand by a polymerase during an amplification reaction. In some embodiments, the mediator probe is protected against (unspecific) polymerase extension by means other than a blocking group (protecting group) at the 3′ end.

In other embodiments, a mediator probe does not comprise a blocking group (protecting group) at the 3′ end and is not protected against (unspecific) polymerase extension.

In embodiments, a mediator probe protected at the 3′ end may comprise a “C3 spacer”. Such a C3 spacer may be a chemical blocking group, which in some embodiments comprises a chain of three carbon atoms. This “C3 spacer” thus preferably prevents (unspecific) polymerase extension of the mediator probe sequence strand. The person skilled in the art is familiar with typical and, depending on the embodiments, suitable blocking groups (protecting groups). Also, based on the present disclosure of the invention, the person skilled in the art knows how to select suitable blocking groups (protecting groups) as routine adaptations of the invention described herein.

Preferably, the method according to the invention thus enables direct endpoint detection of n targets in k detection channels, where n>k and n≥2. In contrast to the prior art, signals according to the invention are not generated by fluorogenic probes, but by target sequence-independent reporter molecules, which can also be described as population-specific reporters, which preferably differ uniquely in the intensity range. This ensures greater flexibility in the experimental design process and allows the use of reporter molecules that utilize contact quenching for initial signal suppression. Furthermore, it allows for controlled signal generation, resulting in more distinguishable and robust fluorescent signals that are particularly suitable for endpoint detection. Preferred embodiments of the present invention are based on universal reporter structures and are used in combination with mediator probes for target sequence detection. One of the basic principles of the method according to the invention is preferably the detection of n target nucleic acid sequences by n target-specific mediator probe types activating n reporter molecule types, wherein each reporter molecule type is labeled with a unique fluorophore with defined optical properties (FIG. 1). In an example of a duplex reaction, two fluorophores would be chosen that emit fluorescence in the same wavelength range but differ in their intensity in the detection range. During dPCR amplification, the bound label-free mediator probes are cleaved by the polymerase and release a mediator sequence. This mediator activates a reporter molecule type, which then generates a fluorescent signal. The sum of all droplets or compartments with a signal of a certain reporter molecule type forms a population in a certain area of the data space, corresponding to the unique fluorescence labeling. Those reporter molecule types labeled with higher quantum yield fluorophores will generate positive populations with higher fluorescence intensities (FIG. 1 at the bottom: high intensities are shown as larger circles), and those with lower quantum yields in the detection region will generate positive populations with lower fluorescence intensities (FIG. 1 at the top: low intensities are shown as smaller circles). Depending on their position in the data space, the populations formed can be assigned to a specific target DNA sequence to be detected in the sample. In the presence of two targets in one compartment, an additive effect of the fluorescence intensities is assumed, which would lead to the formation of a third population with higher signal intensity. In further embodiments, a similar signaling effect could be observed, for example, by using two different quencher types or two similar fluorophore labels on a reporter molecule in a duplex reporter molecule-dPCR reaction. In an example of a four-plex reporter molecule dPCR assay performed in two detection channels, the presence of a plurality of targets in one compartment leads to the formation of orthogonal populations. This means that by using four different reporter molecule types, up to sixteen different populations could theoretically be detected and differentiated (e.g. FIG. 5). Without being bound by theory, it can be assumed that in digital assays the distribution of a target nucleic acid follows Poisson statistics. Thus, the generation of double and multiple positive populations could depend on the concentrations of the target nucleic acid, as well as on possible competing effects caused by the detection of gene loci sharing the same primer pairs. Compared to prior art higher order multiplexing methods, the present method has the advantage that the concentrations of the target sequence-independent reporter molecules can remain constant in different assays. This gives the assay design more freedom and the fluorescence signal populations can be optimized with high efficiency. In SNP detection, another advantage of combining mediator probes with target sequence-independent reporter molecules comes into play. During the extension process, preferably bound mediator probes are cleaved highly specifically by the polymerase, wherein the mediator part is cleaved from the probe part. The cleavage site is preferably located between the first and second nucleotide of the probe part of the mediator probe. Only if the cleavage has been performed correctly, the released mediator can fully bind to the mediator binding site on the specific target sequence-independent reporter molecule type to initiate signal generation. If proper cleavage has not occurred, the existing 3′ overhang will preferably hinder the extension process at the target sequence-independent reporter molecule and no signal will be generated. Therefore, in the case of SNP detection, the first nucleotide may preferably be positioned at the 5′ end of the target-specific probe part of a mediator probe over the SNP position to activate the specific target sequence-independent reporter molecule type.

In a further aspect, the invention relates to a kit comprising:
at least one target sequence-independent reporter molecule of a first type comprising at least one label,
optionally at least one target sequence-independent reporter molecule of a second type comprising at least one label which preferably emits a signal in the same detection channel or the corresponding region of a data space as the at least one target sequence-independent reporter molecule of a first type,
optionally at least one first mediator probe whose mediator sequence has an affinity for the at least one target sequence-independent reporter molecule of a first type, and whose oligonucleotide sequence exhibits an affinity for a first nucleic acid target sequence,
optionally at least one second mediator probe whose mediator sequence has an affinity for the at least one target sequence-independent reporter molecule of a second type, and whose oligonucleotide sequence exhibits an affinity for a second nucleic acid target sequence,
optionally at least one buffer,
optionally a polymerase
optionally a reverse transcriptase
optionally at least one PCR primer.

In one embodiment, the kit according to the invention is suitable or configured for carrying out the method according to the invention.

In embodiments, a kit according to the invention may comprise more than two different types of target sequence-independent reporter molecules and optionally also more than a first and a second mediator probe whose mediator sequence have an affinity for the respective target sequence-independent reporter molecules of a first or second or further type, and whose oligonucleotide sequence correspondingly each has an affinity for a first or a second or further nucleic acid target sequences, such that more than two different nucleic acid target sequences can be specifically detected.

The embodiments described for one aspect of the invention may also be embodiments of any of the other aspects of the present invention. Accordingly, embodiments described for the method according to the invention may also be embodiments of the kit according to the invention. Moreover, any embodiment described herein may also comprise features of any other embodiment of the invention. The various aspects of the invention are unified by, benefit from, are based on, and/or are related to the common and surprising discovery of the unexpected advantageous effects of the present method, namely the optimized simultaneous PCR detection of multiple target sequences by target sequence-independent reporter molecules.

### DETAILED DESCRIPTION OF THE INVENTION

The term “target sequence-independent reporter molecule” describes a molecule or complex of at least one DNA oligonucleotide for signal generation during a PCR in the presence of DNA target sequences. Preferably, a target sequence-independent reporter molecule comprises at least one label and at least one quencher.

In embodiments, a target sequence-independent reporter molecule comprises one or more mediator binding sites. Thus, a target sequence-independent reporter molecule may comprise at least 1, 2, 3, 4, 5, 6, 7, 8, 9, 10, 11, 12, 13, 14, 15, 16, 17, 18, 19, 20, 25, 30, 40 or even 50 mediator binding sites. In preferred embodiments, a target sequence-independent reporter molecule comprises between 1 and 10 mediator binding sites. An (activated) mediator or mediator sequence bound to a mediator binding site, when extended by a (PCR) polymerase, can lead to the activation or degradation, digestion, (de) cleavage or release of one or more labels, e.g. quenchers and/or fluorophores from one or more preferably upstream (towards the 5′ end) modifications located/localized on the reporter molecule.

In the context of the present invention, mediator nucleic acid sequences can be referred to as “mediator probes” or “mediatorprobes”. This always refers to nucleic acid probes which transmit a signal between a target nucleic acid homologous to the mediator probe and a target sequence-independent reporter molecule. During a nucleic acid amplification reaction, e.g. PCR or digital PCR reaction, a polymerase migrating forward on the target nucleic acid can cleave or digest a mediator probe bound to the target nucleic acid. A mediator sequence, or simply “mediator”, is then released, which can bind to a mediator binding site of a target sequence-independent reporter molecule. If a polymerase now also binds to the reporter molecule in the course of PCR and begins to extend the bound mediator sequence, a label is preferably activated, e.g. preferably a quencher is separated from a fluorophore, such that a signal is generated and/or a signal change is effected. In other words, herein a “mediator” or “mediator sequence” refers to a nucleic acid oligonucleotide that can be extended by the polymerase along the target sequence-independent reporter molecule. A “mediator probe” describes a nucleic acid oligonucleotide, preferably a DNA oligonucleotide, which establishes the link between the target sequence and the target sequence-independent reporter molecule by binding to it during a PCR in the presence of a DNA target sequence, is cleaved by the exonuclease activity of the polymerase and releases a mediator sequence which then binds to an associated target sequence-independent reporter molecule for which it has an affinity.

In the context of the present invention, an affinity for a nucleic acid target sequence describes a physical attraction that two homologous nucleic acid sequences have for each other and which can cause them to bind or hybridize to each other. In preferred embodiments, for affinity or successful hybridization of two homologous nucleic acid sequences to each other, there is complete homology between two nucleic acid sequences of a particular length, such as between a probe sequence and a target nucleic acid sequence. In other embodiments, the homologous sequences may comprise at least one mismatch to each other.

In the context of the present invention, a data space is preferably generated by a plot, a diagram or a graph of the different detection channels or by a purely mathematical, algorithm- and/or computer-based evaluation, comparison or juxtaposition of the different detection channels, wherein the respective fluorescence signals of the target sequence-independent reporter molecules used generate data points in this 1-n dimensional data space (depending on the number of detection channels required for the evaluation), which can be grouped into clusters (groups, collections). Thus, in embodiments, a “data space” can be the space which is generated or “spanned” in a plot, diagram or graph preferably by an X-axis and a Y-axis (two-dimensional plot), and optionally—in the case of a three-dimensional graph-additionally by a Z-axis, or in higher dimensions additionally by a further axis, and in which the data to be displayed is represented. In embodiments in which the signals detected in the respective detection channels are displayed on the axes of a graph or plot—for example the signals of the red detection channel on the X-axis, the signals of the green detection channel on the Y-axis and the signals of the blue detection channel on the Z-axis of the plot—the data space can also be described as “spanned by the detection channels”. In further embodiments, a data space is generated by a purely algorithm- and/or computer-based evaluation, comparison and/or juxtaposition of the different detection channels and/or the signals detected therein and is preferably “spanned” by the analyzed detection channels.

By using the method according to the invention, it is possible in embodiments to perform data classification in the higher-dimensional data space (>3 dimensions, with more than three detection channels). In embodiments, the method according to the invention can at least double the number of distinguishable target sequences in a digital PCR compared to the available detection channels, provided that this is not restricted per se by technical limitations on the device side, such as a possible crosstalk between different detection channels.

In the context of the present invention, a “label” may comprise one or more fluorophores and/or one or more quenchers. Accordingly, in the context of the invention, reporter molecules may carry or comprise one or more fluorophores and/or quenchers. Proximity of a fluorophore to the quencher prevents detection of its fluorescence. In embodiments, the reporter-quencher proximity is disrupted, e.g., by release of one or more fluorophores and/or quenchers, wherein in embodiments this is accomplished by partial degradation of the reporter molecule, e.g., at the 5′ end, by hydrolysis by the 5′-to-3′ exonuclease activity of the PCR polymerase used for the amplification reaction. Thus, an unquenched emission of fluorescence is enabled, which can be detected after excitation with a laser. In preferred embodiments, a target sequence-independent reporter molecule comprises at least one fluorophore and at least one quencher (thus these are preferably present “in pairs”), wherein the quencher preferably suppresses the fluorophore signal until a mediator sequence binds to the reporter molecule and is preferably extended by a polymerase as part of a nucleic acid amplification reaction. In embodiments, a spatial separation, e.g. of at least one fluorophore from at least one quencher, is achieved by the polymerase such that a signal is generated and can be detected. In embodiments in which a plurality of quencher-fluorophore pairs are present within a target sequence-independent reporter molecule, the pairs may also be arranged in different localizations relative to each other. A target sequence-independent reporter molecule may comprise one or more labels, for example, there may be at least 1, 2, 3, 4, 5, 6, 7, 8, 9, 10, 11, 12, 13, 14, 15, 16, 17, 18, 19, or 20 labels, or exactly 1, 2, 3, 4, 5, 6, 7, 8, 9, 10, 11, 12, 13, 14, 15, 16, 17, 18, 19, 20, or even 25 labels. In some embodiments, a target sequence-independent reporter molecule comprises 1, 2, 3 or up to 5 labels. In other embodiments, a target sequence-independent reporter molecule comprises 1, 2, 3, 4, 5, or even more than 5 labels.

For the purposes of the invention, a “C3 spacer” is preferably a chemical blocking group (protecting group), which preferably comprises a chain of three carbon atoms. This blocking group (protecting group) preferably serves to prevent (unspecific) polymerase extension of the strand. The person skilled in the art is familiar with typical and—depending on the embodiments-suitable C3 spacers/blocking groups (protecting groups) and the person skilled in the art knows, based on the present disclosure of the invention, how to select suitable C3 spacers/blocking groups (protecting groups) as routine adaptations of the invention described herein.

According to the invention, a signal-generating label is preferably a fluorophore or other dye capable of generating a detectable signal. In some embodiments, a signal generating label is an electroactive or magnetic label. In the context of some embodiments, the terms “signal-generating label” and “label” are equivalent or interchangeable.

In the context of the invention, a biomolecule preferably comprises nucleic acids.

The term “nucleic acid” refers to nucleic acid molecules including, without limitation, DNA, ssDNA, dsDNA, RNA, mRNA, tRNA, lncRNA, ncRNA, microRNA, siRNA, rRNA, sgRNA, piRNA, rmRNA, snRNA, snoRNA, scaRNA, gRNA or viral RNA. Nucleic acid sequences herein refer to a consecutive arrangement of nucleotides, wherein the nucleotides are represented by their nucleobases in guanine (G), adenine (A), cytosine (C) and thymine (T) in DNA and uracil (U) in RNA. A nucleic acid sequence herein may also refer to the sequence of consecutive letters or nucleobases (consisting of G, A, C and T or U) representing the actual sequence of consecutive nucleic acids in a DNA or RNA strand. This nucleic acid sequence can be identified and characterized biochemically and bioinformatically using DNA or RNA sequencing or specifically detected by complementary nucleic acid probes (e.g., in embodiments herein by mediator probes), e.g., as part of a PCR, realtime PCR or detection reaction of a digital PCR. A sequence analysis may also comprise comparing the nucleic acid sequence obtained or a detection signal specific thereto with one or more reference nucleic acid sequences and/or with the detection signals of housekeeping genes. The term nucleotide may be abbreviated as “nt”. The term base pair (two nucleobases bonded to each other via hydrogen bonds) may be abbreviated as “bp”.

In the context of the invention, a “target sequence” (also referred to herein as “target”) describes any nucleic acid sequence of interest to be detected by the method according to the invention. A target sequence may preferably be a DNA, cDNA, cfDNA or RNA sequence. A target sequence may be a part or the entire nucleic acid sequence of a target DNA. A mediator probe preferably comprises a sequence which is wholly or partially complementary to the nucleic acid sequence of the target sequence or a portion thereof. In some embodiments, this mediator probe sequence is 100%, 99%, 95%, 90% or 80% complementary to the target sequence. In some embodiments, a mediator probe can tolerate one or more mismatches to the target sequence and still bind to it. In other embodiments, the mediator probe only binds to a target sequence if it is 100% complementary to the target sequence.

The term “nucleic acid amplification reaction” refers to any process comprising an enzymatic reaction that enables the amplification of nucleic acids. A preferred embodiment of the invention relates to a polymerase chain reaction (PCR). “Polymerase chain reaction” (“PCR”) is the gold standard method for rapidly producing millions to billions of copies (full copies or partial copies) of a given DNA sample, enabling amplification of a very small DNA sample to a sufficiently large amount. PCR amplifies a specific region of a DNA strand (the DNA target sequence) depending on where the primers used bind to start the amplification reaction. Almost all PCR applications use a heat-stable DNA polymerase enzyme, such as Taq polymerase.

In digital PCR (dPCR), the reaction mix and the nucleic acids it contains (e.g. ctDNA or DNA) are divided into thousands of reaction spaces, which separates the target sequences from each other and facilitates their amplification and detection, in particular of rare target sequences. A common method here is the microfluidic dripping of the reaction mix with the nucleic acid to be detected in oil or division into fixed microcavities, whereby each unit formed forms a closed reaction space for amplification and detection, for example during a PCR. If a nucleic acid target sequence is present in a droplet, it is amplified by PCR (or other amplification methods). The individual amplificates are usually detected by sequence-specific fluorogenic nucleic acid probes, wherein corresponding fluorescence signals are generated. In addition to absolute quantification, dPCR also has the advantage that the influence of inhibitors is reduced. Since quantification in digital PCR is performed as an endpoint analysis, a “signal cluster” (also referred to as a population) in the data space can be assigned to a specific DNA or cDNA target sequence via the fluorescence signal strength, which enables an alternative type of data classification (Quan et al. 2018; Whale et al. 2016).

“Multiplexing” generally means detecting a plurality of target sequences simultaneously in one reaction without the need to further divide the reaction mixture for the detection of a plurality of target sequences. In a “multiplex PCR”, a plurality of individual PCR reactions for different DNA sequences or genes are combined under identical conditions to form a single reaction mixture. By allowing a plurality of sequences to be analyzed simultaneously, information can be obtained from a single PCR reaction that would otherwise require many times more reagents and more time to perform. Thus, multiplexing reduces the cost and time required to perform a PCR, as fewer reagents are used per experiment, experiments can be performed faster and results can be analyzed more quickly. Since, by splitting a reaction mixture, a target sequence to be detected may be split into a single detection reaction in which it would not be detected, multiplex detections are more sensitive and are therefore very promising detection methods, particularly in oncological diagnoses. Since pipetting errors can also be minimized in one reaction compared to a plurality of reactions, the use of a multiplex PCR can also improve precision compared to single assays.

Quantitative PCR (“qPCR”) or “real-time PCR” is a specific form of PCR and is a standard method for detecting and quantifying a specific target sequence or quantifying the gene expression level in a sample in real time. In qPCR, fluorescently labeled probes or nucleic acids (e.g., mediator probes) are hybridized in the PCR reaction and, in embodiments, cleaved or digested by the PCR polymerase during primer extension once they bind to a complementary sequence (e.g., a target sequence), wherein, in embodiments, the presence and amplification of target sequences is monitored in real time after or during each PCR cycle. A real-time PCR allows the progress of an ongoing amplification reaction to be monitored as it occurs (i.e., in real time). Data are therefore collected throughout the PCR reaction and not at the endpoint as with conventional PCR. Measuring reaction kinetics in the early stages of PCR offers significant advantages over conventional PCR detection. In embodiments of real-time PCR, reactions are characterized by the time during the cycle when amplification of a target is first detected, rather than by the amount of target that has accumulated after a fixed number of cycles, as in conventional PCR. The higher the starting copy number of the nucleic acid target, the more likely it is that a significant increase in fluorescence will be observed. Real-time PCR enables analysis by means of optical signals that are used to detect a specific PCR product (the target sequence) using specific fluorochromes or fluorophores. An increase in the DNA product during a PCR therefore leads to an increase in the fluorescence intensity measured at each cycle. By using different colored labels, fluorescent probes can be used in multiplex assays to monitor a plurality of target sequences.

While real-time qPCR is dependent on the relative amount of target nucleic acid being determined in each amplification cycle, “digital PCR” allows the absolute amount of target nucleic acid to be determined on the basis of Poisson statistics, which is used to calculate the amount of target nucleic acid following endpoint PCR amplification. The steps prior to amplification are usually comparable or similar between digital PCR and qPCR. However, in qPCR, preferably all nucleic acid molecules are pooled and subsequently amplified and analyzed, whereas in digital PCR, the nucleic acid molecules are preferably divided as best as possible into individual partitions (e.g. emulsion droplets, wells or gel beads), allowing the PCR to proceed as a single reaction in each partition (in the case of emulsion droplets, this reaction is also often referred to as droplet PCR or digital droplet PCR) and allowing separate analysis of each partition. In digital PCR, the random division of the nucleic acid molecules into individual partitions takes place according to the Poisson distribution. When analyzing digital PCR, Poisson statistics are then applied to determine the average number of nucleic acid molecules per partition (none, one or more). Poisson statistical analysis of the number of positive and negative reactions provides a precise absolute quantification of the target sequence.

Recombinase polymerase amplification (“RPA”) is a method for amplifying DNA and is a variant of isothermal DNA amplification. RPA is usually carried out using a recombinase (single-strand-binding protein) and a strand-displacing DNA polymerase, wherein the recombinase increases primer binding. If a strand-displacing polymerase is used, it is possible to carry out the reaction at 37 to 42° C. or even at room temperature. By adding a reverse transcriptase enzyme to an RPA reaction, reverse transcription can be performed. In different variants of RPA, the resulting DNA can optionally be additionally quantified in an RPA reaction (similar to qPCR) and/or several DNA sequences can be amplified in parallel in a multiplex procedure.

The term “loop-mediated isothermal amplification” or “LAMP” refers to a method for amplifying DNA, wherein LAMP is also a variant of isothermal DNA amplification. An isothermal amplification reaction usually takes place at a constant temperature. This property distinguishes LAMP from PCR, in which the reaction takes place with a series of alternating temperature steps or cycles. A strand displacing DNA polymerase is usually used in LAMP. In a LAMP reaction, for example, four to six primers can bind to six to eight DNA sequences, which requires a special primer design. In a LAMP reaction, reverse transcription can be carried out by adding a reverse transcriptase. In addition, the DNA produced in a LAMP reaction can be quantified. It is also possible to detect a plurality of DNA sequences in parallel in a multiplex LAMP reaction.

In the context of the invention, a “signal change” describes a fluorescence signal change. This signal change is preferably a significant, differentiable and/or characteristic change in the fluorescence signal which is clearly distinguishable or differentiated from potential baseline or background signals or baseline or background noise. The person skilled in the art is aware that under some experimental conditions in the context of fluorescence detection, unspecific fluorescence base signals or baseline or background noise can occur due to fluorophores. Therefore, a signal change in the context of the invention preferably describes a significant, differentiable and/or characteristic change in the fluorescence signal, and not a fluorescence base or background signal or baseline or background noise. In preferred embodiments, this signal change can mean an increase in fluorescence intensity, in other words an increase in the fluorescence signal. In some embodiments, a signal change is a decrease in the fluorescence signal. The increase of a fluorescence signal is preferably due to the fact that an amplification reaction increases the number of target sequence amplificates and thus the activation of associated signaling complexes. Accordingly, the number of resulting (de) cleavages, digestions and/or separations of the respective signal oligos from their binding site on the associated base strands increases, whereby at least one fluorophore is released and/or separated from its quencher (i.e. the distance between quencher and fluorophore increases such that the fluorescence signal is no longer quenched by the quencher). An increase (increase in the number) of released and/or non-quenched fluorophores thus leads to an increase in the fluorescence signal, which is specific and indicative of a target sequence. The more target sequences are thus present and are bound by mediator probes in a PCR reaction, the more the fluorescence signal increases. Preferably, the fluorescence signal is proportional or approximately proportional to the amount of the corresponding target sequence for which the fluorophore signal (e.g. its color) is specific/characteristic. Since in the context of a digital or “droplet” PCR preferably only one target sequence is present per reaction space (e.g. partition, emulsion droplet), the signal increases with the number of target sequence amplificates per reaction space until a signal plateau is reached, which is predetermined by the maximum fluorescence intensity of the reporter molecules used for the respective detection. In preferred embodiments, ideally there is a uniform distribution of max. 1 target sequence per reaction space (e.g. partition, emulsion droplets) at the start of amplification such that, with a similar amplification efficiency and similar maximum fluorescence intensity of the detection molecules in all reaction spaces (which contain a target sequence), the specific signal for the detection of an identical target sequence in different reaction spaces with comparable height/strength/intensity is generated during readout by digital or “droplet” PCR, which is preferably indicative of the presence and/or number of target sequence amplificates present in each reaction space. In embodiments, the intensity/strength of a respective label, preferably specific for a target sequence, as well as the maximum achievable signal strength/intensity may depend on the number of labels per signal oligo and signal complex and/or the type of label (e.g. type of fluorophores and/or quenchers).

“Fluorophore” (or fluorochrome, similar to a chromophore) is a fluorescent chemical compound capable of re-emitting light upon light excitation. Fluorophores for use as labels in the design of labeled probes of the invention comprise, without claiming to be exhaustive, rhodamine and derivatives such as Texas Red, fluorescein and derivatives such as 5-bromomethylfluorescein, Lucifer Yellow, IAEDANS, 7-Me2N-coumarin-4-acetate, 7-OH-4-CH3-coumarin-3-acetate, monobromobimane, pyrene trisulfonates such as Cascade Blue and monobromotrimethyl ammoniobimane, 7—NH2-4CH3-25-coumarin-3-acetate (AMCA), FAM, TET, CAL Fluor Gold 540, JOE, VIC, Quasar 570, CAL Fluor Orange 560, Cy3, NED, Oyster 556, TMR, CAL Fluor Red 590, HEX, ROX, LC Red 610, CAL Fluor Red 610, Texas Red, LC Red 610, CAL Fluor Red 610, LC Red 640, CAL Fluor Red 635, Cy5, LC Red 670, Quasar 670, Oyster 645, LC Red 705, Cy5.5, BODIPY FL, Rhodamine Green, Oregon Green 30 488, Oregon Green 514, Cal Gold, BODIPY R6Gj, Yakima Yellow, Cal Orange, BODIPY TMR-X, JOE, HEX, Quasar-570/Cy3, TAMRA, Rhodamine Red-X, Redmond Red, BODIPY 581/591, Cy3.5, Cal Red/Texas Red, BODIPY TR-X, BODIPY 630/665-X, Quasar-670/Cy5, Pulsar-650, Dy490, Atto-488, Atto532, Atto-Rho-6G, Dy590, Atto-Rho101, Cy5, Dy-636, Atto-647N, Cy5.5, Dy682, Atto-680, BMN-488, BMN-505, BMN-536, BMN-562,

“Quenching refers to any process that reduces the fluorescence intensity of a given substance. Quenching is the basis for Forster resonance energy transfer (FRET) assays or static or contact quenching assays or a combination of both. FRET is a dynamic quenching mechanism, as the energy transfer takes place while the donor is in an excited state. Contact quenching requires close proximity in the form of physical contact between donor and quencher. A quencher is a molecule that quenches the fluorescence emitted by the fluorophore when it is excited by the light source of a PCR cycler or detection device. Quenchers for use as labels in the design of labeled signal oligos and/or base strands of the invention comprise, without claiming to be exhaustive, DDQ-I, lowa Black, lowa Black FQ, QSY-9, BHQ-1, QSY-7, BHQ-2, DDQ-II, 22 Eclipse, lowa Black RQ, QSY-21, BHQ-3 Dabcyl, QSY-35, BHQ-0, ElleQuencher, BMN-Q1, BMN-Q2, BMNQ60, BMN-Q-535, BMN-Q590, BMN-Q620, BMN-Q650. The person skilled in the art knows suitable pairs of reporter quenchers and knows which ones to select for a particular application.

### EXAMPLES

The invention is further described by the following examples. These are not intended to limit the scope of the invention, but represent preferred embodiments of various aspects of the invention, which are provided to illustrate the invention described herein.

### Methods

In the following examples, PCR amplifications were carried out in the form of digital PCR. Digital PCR refers to a PCR amplification reaction in which individual nucleic acid molecules are preferably divided into individual partitions (e.g. emulsion droplets, wells or gel beads), whereby the PCR takes place as an individual reaction in each partition (in the case of emulsion droplets, this reaction is also often referred to as droplet PCR or digital droplet PCR) and enables each partition to be analyzed separately. This analysis is preferably performed by fluorescence analysis (e.g. by laser excitation of a fluorophore and detection of its emitted fluorescence) of each individual partition. If a target sequence is located in a partition and is detected by the method according to the invention, this is preferably indicated by a specific fluorescence signal.

The Naica system from Stilla Technologies was used for the listed examples. In the first step, the droplets were generated with the geode and thermally processed. The dPCR protocol used was 5 min at 95° C. followed by 45 cycles of 15 sec at 95° C. and 60 sec at 58° C. The Prism 3 reader and the CrystalMiner software (version 3.1.6.3) developed for this purpose were used to analyze the droplets in examples 1-8 and the Prism 6 reader was used in example 9.

For examples 1-8, single concentrated PerfeCTa® MultiPlex qPCR ToughMix® with 50 nM Alexa Fluor 488 as background fluorophore was used. In duplex and fourplex reactions, 0.5 μM forward and 0.25 μM reverse primers, 1.2 μM mediator probes and 0.6 μM target sequence-independent reporters were used for the respective target sequences. The concentration of target sequence-independent reporters was reduced to 0.4 μM for the sixplex reaction. For example 9, a single concentration of naica® multiplex PCR MIX 10× was used and 1.2 μM mediator probes and 0.4 μM target sequence-independent reporters were used for the respective target sequences as well as 2 μM forward and 1 μM reverse primers overall.

In example 10, the procedure can be analogous to example 9, wherein mediator probes for the ZURs used in example 9 would be adapted to further target sequences.

In order to prevent unspecific extension of relevant oligonucleotide sequences, chemical protecting groups are preferably used to modify the 3′ end of an oligonucleotide sequence. These are, for example, chains comprising three carbon atoms (C3 spacers), which are chemically attached and block the extension of an oligonucleotide sequence by a polymerase during a PCR reaction.

### Sequences:

TABLE 1
Primers

| SEQ | | |
|---|---|---|
| ID | Name | Sequence (5′-3′) |
| 1 | KRAS_forward | GGCCTGCTGAAAATGACT |
| 2 | KRAS_reverse | ACAAAATGATTCTGAATTAGCTGTA |
| 3 | BRAF_forward | GACCCACTCCATCGAGATTTC |
| 4 | BRAF_reverse | GCTTGCTCTGATAGGAAAATGAG |

TABLE 2
Mediator probes. In the sequences, the probe regions are shown in capital
letters; the bases relevant for the point mutation are marked in bold. A C3
spacer is preferably a chemical blocking group comprising a chain of three
carbon atoms to prevent unspecific polymerase extension of the strand.

| SEQ | | | |
|---|---|---|---|
| ID | Name | Sequence (5′-3′) | 3′ modification |
| 5 | MP_ZUR02_KRAS_WT | ctccagttcggtCCAGCTCCAAC- | C3 Spacer |
| | | TACCACAAGTTTATATTCAG | |
| 6 | MP_ZUR04_KRAS_G12A | gatacagggtccaCTGGCG- | C3 Spacer |
| | | TAGGCAAGAGTGCCTTGACG | |
| 7 | MP_ZUR05_KRAS_G12D | atgtcccaggtgcATGGCG- | C3 Spacer |
| | | TAGGCAAGAGTGCCTTGAC- | |
| | | GAT | |
| 8 | MP_ZUR06_KRAS_G12V | aggtaggctcacTTGGCGTAGGG- | C3 Spacer |
| | | CAAGAGTGCCTTGACGAT | |
| 9 | MP_ZUR07_BRAF_V600E | acatgctcatgttgtgTCTGTAGCTA- | C3 Spacer |
| | | GACCAAAATCAC- | |
| | | CTATTTTTACTGTGAG | |
| 10 | MP_ZUR08_BRAF_WT | gtgttcctcacatgctACTGTAGCTA- | C3 Spacer |
| | | GACCAAAATCAC- | |
| | | CTATTTTTACTGTGAG | |
| 23 | MP_ZUR01_KRAS_G12D | ggctctacgaccaacATGGCG- | C3 Spacer |
| | | TAGGCAAGAGTGCCTTGAC- | |
| | | GAT | |

TABLE 3
Target sequence-independent reporter molecules (The fluorophore modifications are
located internally at the first 3′ base of the stem-loop structure (nt 22 (7; 6) or nt
20 (8); shown as 6, 7 or 8); quenchers were attached to the first base of the stem-loop
structure on the 5′ side (nt 1)). A C3 spacer is preferably a chemical blocking group
comprising a chain of three carbon atoms to prevent unspecific polymerase extension of
the strand.

| | | | | | Internal |
|---|---|---|---|---|---|
| SEQ | | | 5′-mod. | | modification |
| ID | Name | Sequence (5′-3′) | quencher | 3′ modification | of fluorophore |
| 11 | ZURO2 | GACCGGCCAAGAC- | BMN-Q1 | C3 Spacer | 7 = dC-DY-530 |
| | | GCGCCGGT7TGTTCAC- | | | |
| | | TGACCGAACTGGAGCA | | | |
| 12 | ZURO4 | GACCGCACTAG- | BHQ-1 | C3 Spacer | 7 = dC-BMN536 |
| | | TAGATGCGGT7TGTCGTG | | | |
| | | GACCCTGTATCGAGCA | | | |
| 13 | ZURO5 | GACCGGCTAAGAC- | BHQ-2 | C3 Spacer | 7 = dC-Cy5 |
| | | GCGCCGGT7TGTTGCAC- | | | |
| | | CTGGGACATCGACTAT | | | |
| 14 | ZURO6 | GACGCGTAGTACAGAAC- | BHQ-2 | C3 Spacer | 7 = dC-Atto 647N |
| | | GCGT7TGTTCAG- | | | |
| | | TGAGCCTACCTGCCTTC | | | |
| 15 | ZURO7 | ATGCCGATTAGATGCGG | BHQ-1 | C3 Spacer | 8 = dT-FAM |
| | | CA8TCGATCACACAACAT | | | |
| | | GAGCATGTGTAC | | | |
| 16 | ZURO8 | ATCCGCCAAGAC- | BMN-Q1 | C3 Spacer | 8 = dT-Atto 488 |
| | | GCGCGGA8TAG- | | | |
| | | CATGTGAGGAACAC- | | | |
| | | GATGACAC | | | |
| 24 | ZURO1 | GACCGGCCAAGAC- | BHQ-2 | C3 Spacer | 7 = dC-Cy5.5 |
| | | GCGCCGGT7TGTT- | | | |
| | | GGTCG- | | | |
| | | TAGAGCCCAGAACGA | | | |
| 25 | ZURO2.2 | GACCGGCCAAGAC- | BHQ-2 | C3 Spacer | 6 = dC-Dy636 |
| | | GCGCCGGT6TGTTCAC- | | | |
| | | TGACCGAACTGGAGCA | | | |
| 26 | ZURO6.2 | GACGCGTAGTACAGAAC- | BHQ-2 | C3 Spacer | 7 = dC-Atto680 |
| | | GCGT7TGTTCAG- | | | |
| | | TGAGCCTACCTGCCTTC | | | |

TABLE 4
Target sequences (primer hybridization regions are underlined; point mutations/
SNPs are shown in bold)

| SEQ | | |
|---|---|---|
| ID | Name | Sequence (5′-3′) |
| 17 | BRAF | CAAACTGATGGGACCCACTCCATCGAGATTTCACTGTAGCTAGAC- |
| | WT | CAAAATCAC- |
| | | CTATTTTTACTGTGAGGTCTTCATGAAGAAATATATCTGAGGTAGTAGTAAG |
| | | TAAAGGAAAACAGTAGATCTCATTTTCCTATCAGAGCAAGCATTATGAA- |
| | | GAGTTT |
| 18 | BRAF | CAAACTGATGGGACCCACTCCATCGAGATTTCTCTGTAGCTAGAC- |
| | V600E | CAAAATCAC- |
| | | CTATTTTTACTGTGAGGTCTTCATGAAGAAATATATCTGAGGTGTAGTAAGT |
| | | AAAGGAAAACAGTAGATCTCATTTTCCTATCAGAGCAAGCATTATGAAGAG- |
| | | TTT |
| 19 | KRAS | CATTTTCATTATTTTTATTATAAGGCCTGCTGAAAATGACTGAATATAAACTT- |
| | WT | GTGGTAGTT- |
| | | GGAGCTGGTGGCGTAGGCAAGAGTGCCTTGACGATACAGCTAATTCAGAA |
| | | TCATTTTGTGGACGAATAT |
| 20 | KRAS | CATTTTCATTATTTTTATTATAAGGCCTGCTGAAAATGACTGAATATAAACTT- |
| | G12A | GTGGTAGTT- |
| | | GGAGCTGCTGGCGTAGGCAAGAGTGCCTTGACGATACAGCTAATTCAGAA |
| | | TCATTTTGTGGACGAATAT |
| 21 | KRAS | CATTTTCATTATTTTTATTATAAGGCCTGCTGAAAATGACTGAATATAAACTT- |
| | G12D | GTGGTAGTT- |
| | | GGAGCTGATGGCGTAGGCAAGAGTGCCTTGACGATACAGCTAATTCAGAA |
| | | TCATTTTGTGGACGAATAT |
| 22 | KRAS | CATTTTCATTATTTTTATTATAAGGCCTGCTGAAAATGACTGAATATAAACTT- |
| | G12V | GTGGTAGTT- |
| | | GGAGCTGTTGGCGTAGGCAAGAGTGCCTTGACGATACAGCTAATTCAGAAT |
| | | CATTTTGTGGACGAATAT |

### Example 1

In this example, a signal is generated when a mediator probe binds to a nucleic acid target sequence and is digested or cleaved by a polymerase, which amplifies a nucleic acid sequence, as part of a PCR reaction. The mediator, or mediator sequence, is thus released and can bind to a target sequence-independent reporter molecule. If the bound mediator is now recognized by a polymerase as a primer or start oligonucleotide for amplification during the PCR reaction, bound and extended by the polymerase, a quencher or fluorophore located “downstream” (downstream of the amplification direction) of the mediator binding site is separated or displaced, such that a signal is generated by a fluorophore-previously quenched by contact quenching. This signal is characteristic of the nucleic acid target sequence to which the mediator probe has previously bound. The method according to the invention enables (direct) monochrome multiplexing in digital PCR by using at least two target sequence-independent reporter molecules (see e.g. “target sequence-independent reporter” in FIG. 1), each with different fluorophore modifications (see e.g. FIG. 1, shown therein as a circle or triangle) with an emission maximum in one (e.g. in the red) detection channel in a configuration that enables signal identification. In FIG. 1, different signal strengths of the fluorescence are represented by circles of different sizes. By using target sequence-independent reporter molecules, in contrast to target sequence-specific Taqman probes of the prior art, contact quenching can be adjusted more efficiently, whereby the differences in the signal strength of different fluorophores in the same detection channel can be amplified or modeled more easily.

For (direct) multiplexing, i.e. the simultaneous detection of different target sequences in a sample to be analyzed and/or in a reaction, a plurality of optical channels, further process steps or a complex concentration adjustment or modification of the reporter molecules are usually required in the prior art. In contrast to this, the method according to the invention makes it possible to detect different target sequences in a sample in the same detection channel without being limited by the points listed above. The use of target sequence-independent reporter molecules also enables the reuse of optimized designs for different target sequences and makes their application correspondingly more efficient. For example, different intensity combinations and/or different fluorophore colors of the respective labels, preferably different extended mediator sequences and thus different detected target sequences can be encoded and differentiated.

In embodiments, a plurality of target sequences can be detected simultaneously via different fluorescence intensities and/or fluorescence colors by using different target sequence-independent reporter molecules. These target sequence-independent reporter molecules each have different mediator binding sites with fluorescence and/or quencher labels of different signal intensity and/or color/emission spectrum. Preferably, however, target sequence-independent reporter molecules are used, of which at least two reporter molecules can always generate signals that can be measured/detected in the same detection channel.

By generating signals with different signal strengths, these can be distinguished in one fluorescence channel. These embodiments offer considerable advantages, particularly in digital PCR, for making different signal clusters distinguishable and represent an improvement on the prior art.

### Example 2

An example in which two target sequence-independent reporter molecules with different fluorophore and quencher numbers and an emission maximum in the red detection channel are used is another example of (direct) monochrome multiplexing according to the present invention. In this example, different fluorescence signal strengths are generated by different numbers of fluorophores during digital PCR, thus enabling signal identification. By using target sequence-independent reporter molecules, e.g. in contrast to target sequence-specific Taqman probes, symmetric multiple modification can be used more efficiently on fluorophores and quenchers, thereby amplifying the differences in signal strength of different fluorophores in the same detection channel.

### Example 3

In one sample analysis, (direct) monochrome multiplexing was performed by using 2 target sequence-independent reporter molecules with different fluorophore labels in the red detection channel. Here, the two different target sequence-independent reporter molecules were labeled with either Cy5 or Atto-647N. The analysis comprised an amplification reaction in the context of a digital PCR (Mediator Probe PCR) in the Stilla Naica system, wherein the results obtained can be displayed graphically in a plurality of dimensions of a data space.

FIG. 2 shows a graphical evaluation and representation of the digital PCR results in the form of a 1-D (one-dimensional) plot.

FIG. 3 shows a graphical evaluation and representation of the digital PCR results in the form of a 2-D (two-dimensional) plot. Here, the specific signals of the reporter complexes/reporter molecules in the red channel (“red”, Y-axis) were compared with the background signal (background noise) in the blue detection channel (“blue”, X-axis) in a two-dimensional data space. The two different signals of the reporter complexes/reporter molecules can be easily differentiated from each other as distinct signal populations in the two-dimensional data space and from the background noise (in the blue detection channel).

FIG. 4 shows a graphical evaluation and representation of the digital PCR results in the form of a 3-D (three-dimensional) plot. Here, the specific signals of the reporter complexes/reporter molecules in the red channel (“red”, Y-axis) were compared with both the background signal in the blue detection channel (“blue”, X-axis) and the signals of a negative control (sample without target gene) in the green channel (“green”, Z-axis) in a three-dimensional data space. In this example, the two different signals of the reporter complexes/reporter molecules can also be easily differentiated from each other as distinct signal populations in the three-dimensional data space and from the background noise and a signal of the negative control—not detected here, but potentially present in other analyses.

### Example 4

In the present experiment, (direct) monochrome multiplexing in two detection channels (e.g. red and green channel) is performed in a digital PCR reaction using mediator probes by using four different types of target sequence-independent reporter molecules (ZUR1-ZUR4) with different fluorescent labels. In a graphical representation of the signals detected during the digital PCR reaction, which are generated by the different types of reporter molecules, these can be represented in the form of signal populations in the data space (the space that is generated or spanned by an X-axis and a Y-axis and in which the data are graphically represented).

FIG. 5 shows a theoretical schematic of the representation of an analysis of signals from an example with four different reporter molecules (ZUR1-ZUR4) wherein different florescence signal intensities are represented by different sized circles and positions in the data space. The data space in FIG. 5 is spanned by the X-axis, on which the intensity of detected signals in the red detection channel (ZUR4 and ZUR2) is shown, and the Y-axis, on which the detected signal intensity in the green detection channel (ZUR3 and ZUR1) is shown. The signal populations in the data space can be clearly assigned to one of the four types of activated target sequence-independent reporter molecules and could therefore be efficiently used to detect different target sequence panels (target sequence sets or combinations) in contrast to target sequence-specific reporter molecules (e.g. Taqman probes).

### Example 5

In a further example of the invention, samples in the red, green and blue detection channel could be analyzed in parallel during a (direct) monochrome mediator probe multiplex PCR. For this purpose, the samples were amplified and analyzed in a corresponding digital PCR system, such as the Stilla Naica system. For each detection channel, two, one and no DNA target sequences were added to a duplex reaction (reaction mixture comprising two different reporter molecule types, each specific for one of the two DNA target sequences), such that either both target sequences could be detected simultaneously or only one or none of them could be detected in the respective detection channel.

FIG. 6 shows the results of this example for the use of two different reporter complexes/reporter molecules in the red detection channel in a (direct) monochrome multiplexing analysis in the Stilla Naica system. The signals of a duplex reaction (double reaction) are shown, wherein the signals characteristic of target sequence 1 and target sequence 2 (X-axis) are shown in the leftmost data space. In the middle data space, the signals of the reactions with single addition of a target sequence are shown (single reactions in the duplex reaction approach), which are specific for the individual target sequence 1 (X-axis) or target sequence 2 (X-axis). The measured signal for the negative control (NTC; X-axis) is shown in the rightmost data space. The Y-axis shows the detected signal intensity in the red detection channel. Two types of target sequence-independent reporter molecules (ZUR05 and ZUR06), either labeled with Cy5/BHQ-2 or BHQ-2/Atto-647N, were used for signal generation.

The different signals of the reporter complexes/reporter molecules can be easily differentiated from each other as distinct signal populations in the two-dimensional data space, such that specific detection of both target sequences was possible in each case.

FIG. 7 shows the results of this example for the use of two different reporter complexes/reporter molecules in the green detection channel in a (direct) monochrome multiplexing analysis in the Stilla Naica system. The signals of a duplex reaction (double reaction) are shown, wherein the detected signals specific for target sequence 1 and target sequence 2 (X-axis) are shown in the leftmost data space. The two individual detections after the addition of only one DNA target sequence each for the individual detection of target sequence 1 (X-axis) and target sequence 2 (X-axis) are shown in the middle data space. The negative control (NTC; X-axis) is shown in the rightmost data space. The signal intensity in the green detection channel is shown on the Y-axis. Two types of target sequence-independent reporter molecules (ZUR02 and ZUR04) were used for signal generation, either labeled with Dy530 and BMN-Q1 or with BMN536 and BHQ-1.

The different signals of the reporter complexes/reporter molecules can be easily differentiated from each other as distinct signal populations in the two-dimensional data space, such that specific detection of both target sequences was possible in each case.

FIG. 8 shows the results of this example for the use of two different reporter complexes/reporter molecules in the blue detection channel in a (direct) monochrome multiplexing analysis in the Stilla Naica system. The signals of a duplex reaction (double reaction) are shown, wherein the signals specific for target sequence 1 and target sequence 2 (X-axis) are shown in the leftmost data space. The two individual detections after the addition of only one DNA target sequence each, which indicate the presence of the individual target sequence 1 (X-axis) or target sequence 2 (X-axis), are shown in the middle data space. The signal of the negative control (NTC; X-axis) is shown in the rightmost data space. The signal intensity in the blue detection channel is shown on the Y-axis. Two types of target sequence-independent reporter molecules (ZUR07 and ZUR08), labeled with either FAM/BHQ-1 or Atto488/BMN-Q1, were used for signal generation.

The different signals of the reporter complexes/reporter molecules can be easily differentiated from each other as distinct signal populations in the two-dimensional data space, such that specific detection of both target sequences was possible in each case.

### Example 6

In a further example of the invention, during a (direct) monochrome mediator probe multiplex PCR, samples in the green and blue detection channel could each be analyzed for the presence of 4 target sequences simultaneously. For this purpose, the samples were amplified and analyzed in a corresponding digital PCR system, such as the Stilla Naica system. In this “4-Plex Mediator Probe PCR”, four target sequence-independent reporter molecules and four target sequence-specific mediator probes were provided to detect the DNA target sequences I-KRAS G12A (KRAS gene mutation G12A), II-KRAS WT (KRAS wild-type gene), III-BRAF V600E (BRAF gene mutation V600E) and IV-BRAF WT (BRAF wild-type gene). The reporter molecules for KRAS target gene variants (different BRAF or KRAS single nucleotide polymorphisms (SNP) or wild-type (WT) variants) generated a signal in the green detection channel (BMN536 and Dy-530 fluorophore) upon activation, the reporter molecules for BRAF target gene variants generated a signal in the blue detection channel (FAM and Atto 488 fluorophore) upon activation.

FIG. 9 shows the result of this SNP analysis, wherein the signals of target sequences I and II detected individually in a partition are shown in the upper left quadrant of the data space. The signals of target sequences III and IV detected individually in a partition are shown in the lower right quadrant of the data space. The signals of one KRAS and one BRAF target sequence (i.e. I+III, II+III, I+IV and II+IV) detected simultaneously in one partition are shown in the upper right quadrant of the data space. The signal intensity in the green detection channel (BMN536 and Dy530 fluorophore) is shown on the Y-axis and the signal intensity in the blue detection channel (FAM and Atto 488 fluorophore) on the X-axis.

As can be seen in FIG. 9, a plurality of signal clusters can be detected per detection channel, which can be assigned to activated, target sequence-independent reporter molecules and thus to the corresponding target sequences.

### Example 7

In a further example of the invention, during a (direct) monochrome mediator probe multiplex PCR, samples in the green, red and blue detection channel could be analyzed for the presence of 6 target sequences simultaneously. For this purpose, the samples were amplified and analyzed in a corresponding digital PCR system, such as the Stilla Naica system. In this “6-Plex Mediator Probe PCR”, six target sequence-independent reporter molecules and six target sequence-specific mediator probes were provided to analyze the target sequences I-BRAF V600E (BRAF gene mutation V600E), II-BRAF WT (BRAF wild-type gene), III-KRAS WT (KRAS wild-type gene), IV-KRAS G12A (KRAS gene mutation G12A), V-KRAS G12D (KRAS gene mutation G12D), VI-KRAS G12V (KRAS gene mutation G12V). The reporter molecules for BRAF target gene variants BRAF V600E and WT generated a signal in the blue detection channel (FAM or Atto 488 fluorophore) upon activation, the reporter molecules for KRAS WT and G12A target gene variants generated a signal in the green detection channel (DY-530 or BMN-536 fluorophore) upon activation and the reporter molecules for KRAS G12D and G12V target gene variants generated a signal in the red detection channel (Cy5 and Atto-647N fluorophore) upon activation.

FIG. 10 shows the result of this analysis, wherein the signals of target sequences I and II detected individually in a partition are shown on the X-axis of the data space. The signals of target sequences V and VI detected individually in a partition are shown on the Y-axis of the data space. The signals of target sequences III and IV detected individually in a partition are shown on the Z-axis of the data space. The signals of the respective KRAS or BRAF target sequences detected simultaneously in a partition are also shown in the data space. The signal intensity in the red detection channel is shown on the Y-axis, the signal intensity in the blue detection channel on the X-axis and the signal intensity in the green detection channel on the Z-axis.

As can be seen in FIG. 10, a plurality of signal clusters can be detected per detection channel, which can be assigned to activated, target sequence-independent reporter molecules and thus to the corresponding target sequences.

### Example 8

By using the method described, it is also possible to perform data classification in higher-dimensional data space (>3 dimensions with more than three detection channels). Using the method described, the number of distinguishable target sequences in a digital PCR can theoretically be at least double the number of available detection channels, provided this is not restricted per se by technical limitations on the device side such as crosstalk between different detection channels:

| Number of detection channels | Theoretical minimum number of |
|---|---|
| on the device side | differentiable target sequences |
| 1 | 2 |
| 2 | 4 |
| 3 | 6 |
| 4 | 8 |
| 5 | 10 |
| 6 | 12 |
| 7 | 14 |
| N | 2N |

The person skilled in the art is familiar with corresponding methods for classifying higher-dimensional data structures.

### Example 9

In a further example, the multiplexing capacities can be increased beyond doubling for each detection channel using the method described. Thus, multiplexing capacities can be at least tripled or further increased per detection channel. This is made possible by using at least three reporter molecules in a detection channel, which generate at least three distinguishable populations in the data space in the respective detection channel, which are specifically assigned to each reporter.

This allows up to three target sequences to be detected in one detection channel, which triples the device-side detection channel capacities, provided they are not restricted per se by device-side technical limitations such as crosstalk between different detection channels. FIG. 12 shows the results of such a tripling of the detection channels in the Stilla Prism-6. Here, the presence of three target sequences was analyzed simultaneously in the infrared detection channel of the Stilla Prism6. For this purpose, the samples were amplified and analyzed in a corresponding digital PCR system, such as the Stilla Naica system. In this “3-Plex Mediator Probe PCR”, three target sequence-specific mediator probes were provided to detect the three target sequences I-KRAS G12D (KRAS single point mutation G12D), II KRAS WT (KRAS wild-type genome sequence) and III KRAS G12V (KRAS single point mutation G12V) by means of the three target sequence unspecific reporter molecules with the fluorophore quencher combinations Cy5.5+BHQ2 (I KRAS G12D), Dy636+BHQ2 (II KRAS WT) and Atto680+BHQ2 (III KRAS G12V). A particular advantage of this method over the prior art is that signal generation using target sequence-unspecific reporter molecules makes signal generation very robust and populations are generated in the data space at comparable distances from each other across a plurality of independent experiments, thus simplifying data analysis. This increases the benefit of the signal optimization required for such population separation.

### Example 10

In a further example, due to the separation of the detection of one or more different DNA target sequences by the cleavage of one or more different mediator probes combined with the release of one or more different mediator sequences and signal generation by the extension of one or more mediator sequences on one or more different target sequence-independent reporter molecules, a set of target sequence-independent reporter molecules can be used for the detection of different panels of DNA target sequences. In this case, the target sequence-independent reporter molecules remain constant and are adapted to the different DNA target sequences by exchanging the mediator probes and/or primers. In this way, the efficiency of the optimization of fluorescence signals of the target sequence-independent reporters is much higher, since these would only have to be optimized once for each detection device (for example a digital PCR thermal cycler) compared to the prior art. This means that signal optimization can be used to generate many different, highly differentiable fluorescence signal populations in the data space more efficiently than before, as this optimization only has to be carried out once and can then be transferred to other DNA target sequence panels. Corresponding optimization processes are therefore more efficient and are generally known to the person skilled in the art and described in the specialist literature (Lehnert et al 2018). This means that, due to the optimized signals, assays that use direct monochrome multiplexing can achieve better performance in the areas of sensitivity, precision or specificity with less effort than prior art assays whose signals have to be optimized again for each new target sequence. Compared to the prior art, this has the particular advantage that a signal can always be clearly assigned to the same reporter by its position in the data space alone and that it is not necessary to correlate this position in the data space with other information, for example the temperature or the position of other signal populations in the data space, in order to determine the activation of the respective target sequence-unspecific reporter.

### FIGURES

The invention is further described by the following figures. These are not intended to limit the scope of the invention, but represent preferred embodiments of aspects of the invention, provided to illustrate the invention described herein.

FIG. 1: Direct monochrome multiplexing in digital PCR by using two target sequence-independent reporter molecules (“ZUR”) of a first (A) and a second (B) type, each with different fluorophore labeling (shown as a circle or triangle) with an emission maximum in the red detection channel in a configuration that enables signal identification. Different signal strengths of the fluorescence, which are characteristic of a respective nucleic acid target sequence, are represented by circles of different sizes. By using target sequence-independent reporter molecules, in contrast to target sequence-specific Taqman probes, contact quenching (of at least one fluorophore by at least one quencher) can be adjusted more efficiently, whereby the differences in the signal strength of different fluorophores in the same detection channel are amplified. The at least one quencher is localized at the 5′-end of the reporter molecule (“5′-Quencher modification”). Each type of reporter molecule (“ZUR 1” under (A) or “ZUR 2” under (B)) is bound and activated by a specific mediator sequence (“Mediator 1” under (A) or “Mediator 2” under (B)), which was previously cleaved and/or released from the corresponding mediator probe (“Mediator Probe 1” under (A) or “Mediator Probe 2” under (B)).

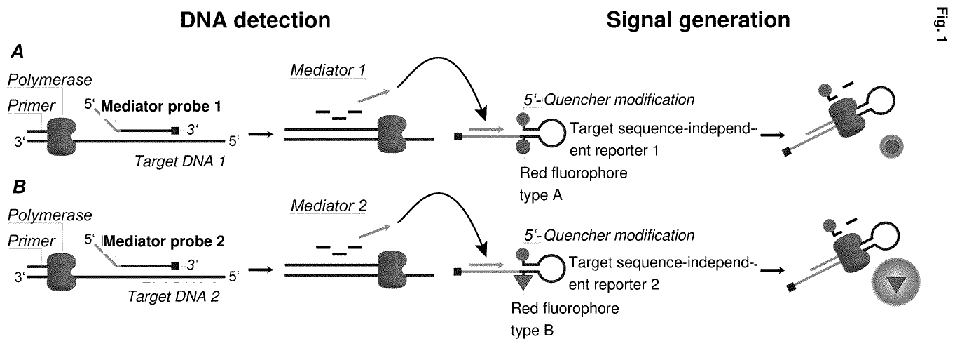

FIG. 2: The graph represents the results of example 3 by a one-dimensional plot (1-D plot). In example 2, two nucleic acid target sequences were detected by a (direct) monochrome (“mediator probe”) duplex PCR (digital PCR in the Stilla Naica system) using associated specific target sequence-independent reporter molecule types of a first and a second type. The two different reporter molecule types comprised either the red fluorophore Cy5 or the red fluorophore Atto 647N as labels, which were characteristic of the respective nucleic acid target sequence and were detected and analyzed in the red detection channel of a Stilla Naica system (digital PCR system).

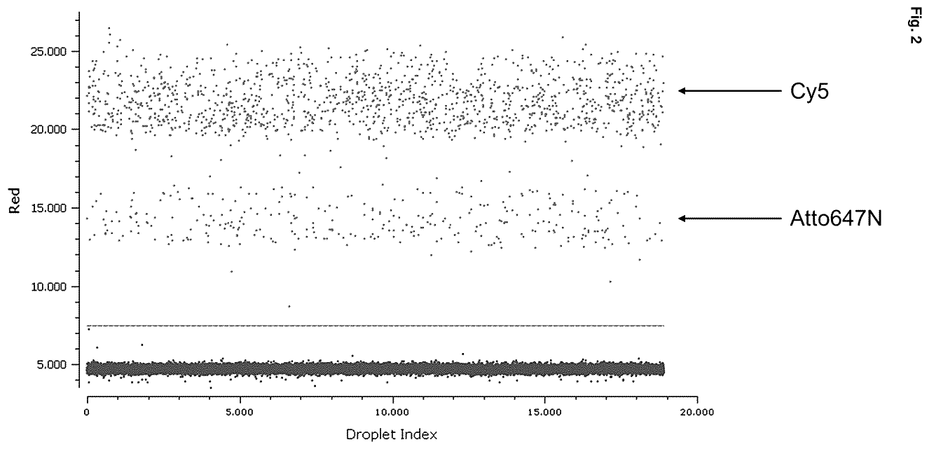

FIG. 3: The graph represents the results of example 3 by a two-dimensional (2-D) plot. In example 2, two nucleic acid target sequences were detected by (direct) monochrome (“mediator probe”) duplex PCR (digital PCR in the Stilla Naica system) using associated specific target sequence-independent reporter molecule types of a first and a second type. The two different reporter molecule types comprised either the red fluorophore Cy5 or the red fluorophore Atto 647N as labels, which were characteristic of the respective nucleic acid target sequence and were detected in the red detection channel of a Stilla Naica system (digital PCR system). This plot compares the specific signals of the reporter complexes/reporter molecules in the red channel (“Red”, Y-axis) with the background signal (background noise) in the blue detection channel (“Blue”, X-axis) in a 2-D data space. Unit of the Y-axis: relative fluorescence units (RFU).

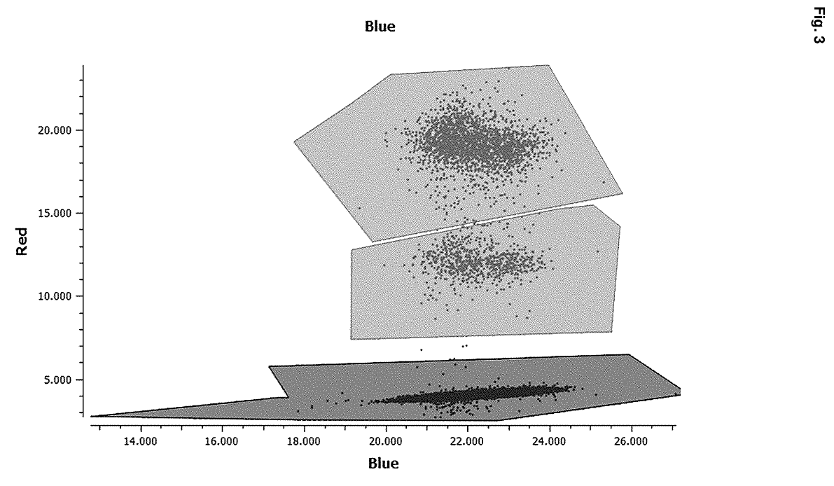

FIG. 4: The graph represents the results of example 3 by a three-dimensional (3-D) plot. In example 2, two nucleic acid target sequences were detected by (direct) monochrome (“mediator probe”) duplex PCR (digital PCR in the Stilla Naica system) using associated specific target sequence-independent reporter molecule types of a first and a second type. The two different reporter molecule types comprised either the red fluorophore Cy5 or the red fluorophore Atto 647N as labels, which were characteristic of the respective nucleic acid target sequence and were detected in the red detection channel of a Stilla Naica system (digital PCR system). This plot compares the specific signals of the reporter complexes/reporter molecules in the red channel (“Red”, Y-axis) with the background signal (background noise) in the blue detection channel (“Blue”, X-axis) and the signals of a negative control (contained no target sequence) in the green detection channel (“Green”, Z-axis) in a 3-D data space. Unit of the axes: relative fluorescence units (RFU).

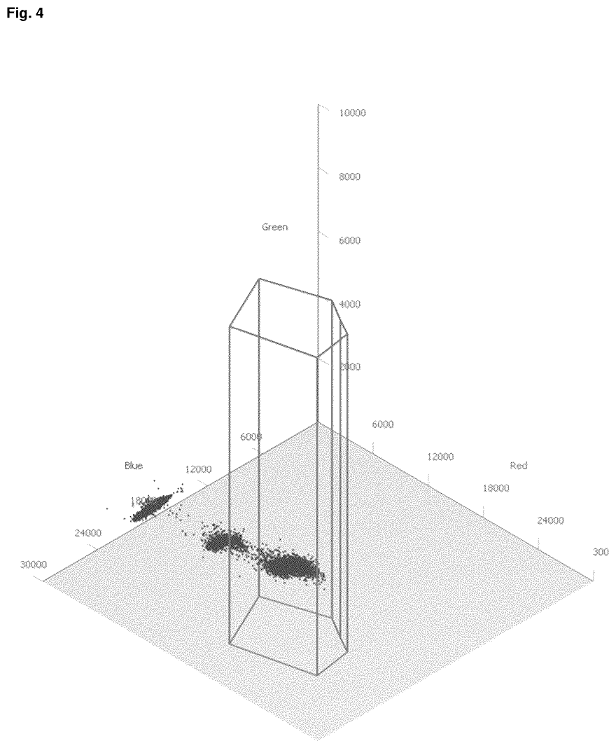

FIG. 5: A schematic of the test design and the reporter molecules used in example 4 is shown. In example 4, direct monochrome multiplexing in two detection channels (e.g. red channel (X-axis) and green channel (Y-axis)) is performed in a digital PCR reaction using four different types of target sequence-independent reporter molecules (ZUR1-ZUR4) with different fluorescent labels. The four different types of target sequence-independent reporter molecules (ZUR1-ZUR4) are each bound and activated by a specific mediator sequence (mediator type 1-4), wherein the generated and detected fluorescence signal is specific for a respective nucleic acid target sequence.

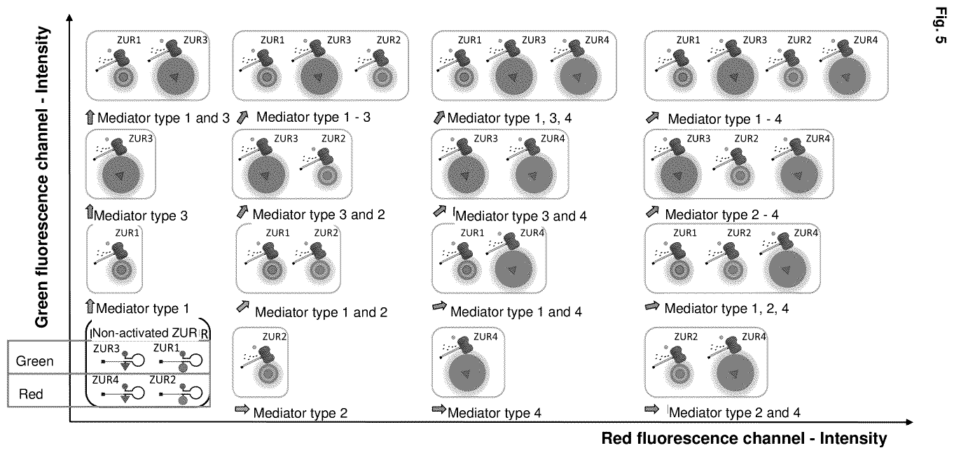

FIG. 6: The results of example 5 are shown. Two different target sequence-independent reporter molecule types were used in the red detection channel in a (direct) monochrome multiplexing PCR analysis in the Stilla Naica system. The signals of a duplex reaction (double reaction) are shown, wherein the signals indicating the presence of target sequence 1 and target sequence 2 (X-axis) are shown in the leftmost data space. The signals of the individual detections (after addition of only one DNA target sequence each), which are specific for target sequence 1 (X-axis) and target sequence 2 (X-axis), are shown in the middle data space. The signal of the negative control (NTC; X-axis) is shown in the rightmost data space. The signal intensity in the red detection channel (“Red”) is shown on the Y-axis. Two types of target sequence-independent reporter molecules (ZUR05 and ZUR06), either labeled with Cy5/BHQ-2 or BHQ-2/Atto-647N, were used for signal generation. Unit of the Y-axis: relative fluorescence units (RFU).

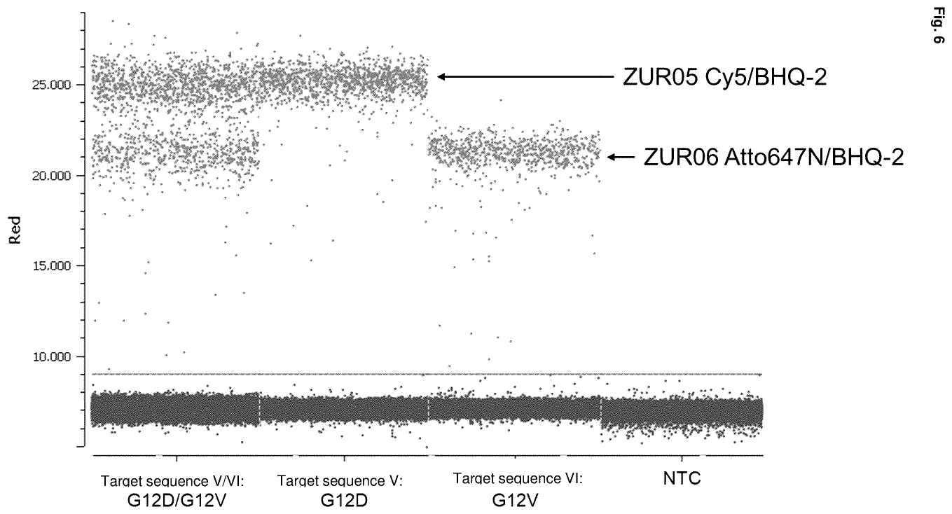

FIG. 7: The results of this example 5 are shown for the use of two different reporter complexes/reporter molecules in the green detection channel in a (direct) monochrome multiplexing analysis in the Stilla Naica system. The signals of a duplex reaction (double reaction) are shown, wherein the signals specific for target sequence 1 and target sequence 2 (X-axis) are shown in the leftmost data space. The two individual detections after the addition of only one DNA target sequence each for the detection of the individual target sequence 1 (X-axis) or target sequence 2 (X-axis) are shown in the middle data space. The signal of the negative control (NTC; X-axis) is shown in the rightmost data space. The signal intensity in the green detection channel is shown on the Y-axis. Two types of target sequence-independent reporter molecules (ZUR02 and ZUR04), either labeled with Dy530/BMN-Q1 or with BMN536/BHQ-1, were used for signal generation. Unit of the Y-axis: relative fluorescence units (RFU).

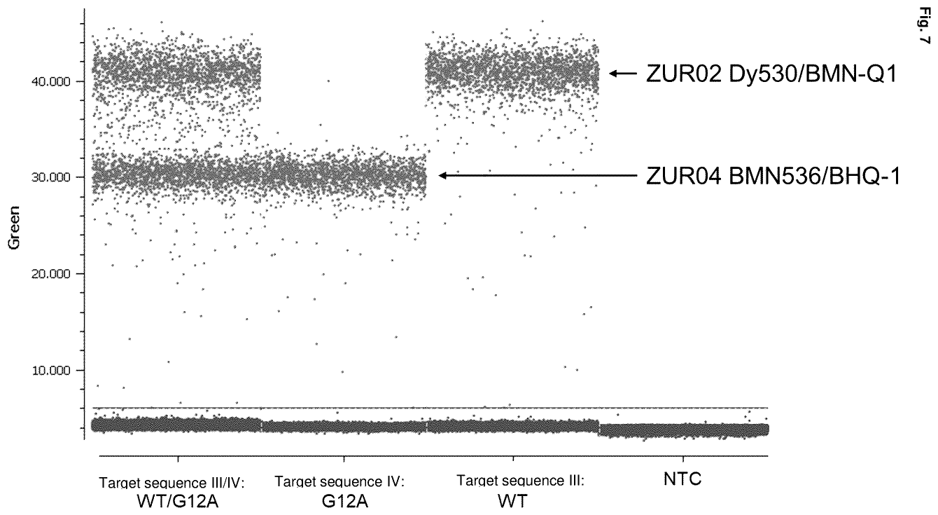

FIG. 8: The results of this example 5 are shown for the use of two different reporter complexes/reporter molecules in the blue detection channel in a (direct) monochrome multiplexing analysis in the Stilla Naica system. The signals of a duplex reaction (double reaction) are shown, wherein the signals specific for target sequence 1 and target sequence 2 (X-axis) are shown in the leftmost data space. In the middle data space, the individual detections after the addition of only one DNA target sequence each are shown, which indicate the presence of the individual target sequence 1 (X-axis) or target sequence 2 (X-axis). The signal of the negative control (NTC; X-axis) is shown in the rightmost data space. The signal intensity in the blue detection channel is shown on the Y-axis. Two types of target sequence-independent reporter molecules (ZUR07 and ZUR08), either labeled with FAM/BHQ-1 or with Atto488/BMN-Q1, were used for signal generation. Unit of the Y-axis: relative fluorescence units (RFU).

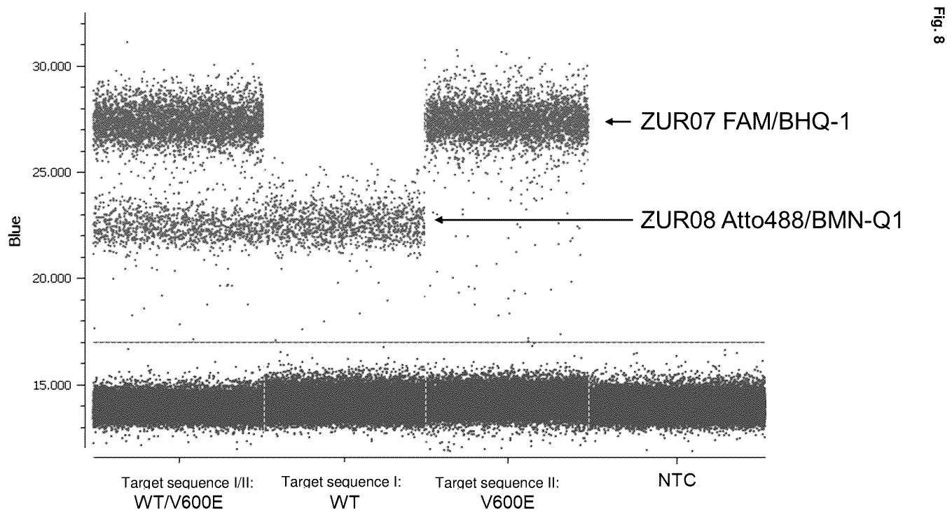

FIG. 9: The result of example 6 is shown, wherein samples in the green and blue detection channel were analyzed for the presence of 4 target sequences simultaneously using a (direct) monochrome mediator probe multiplex PCR. For this purpose, the samples were amplified and analyzed in a corresponding digital PCR system (Stilla Naica system). In this “4-Plex Mediator Probe PCR”, specific target sequence-independent reporter molecules were used for the target sequences I-KRAS G12A (KRAS gene mutation G12A), II-KRAS WT (KRAS wild-type gene), III-BRAF V600E (BRAF gene mutation V600E) and IV-BRAF WT (BRAF wild-type gene). The reporter molecules for KRAS target gene variants generated a signal in the green detection channel (Y-axis) upon activation, the reporter molecules for BRAF target gene variants generated a signal in the blue detection channel (X-axis) upon activation. In the depicted 2-D plot (2-D data space), the signals of target sequences I and II detected individually in a partition are shown in the upper left quadrant of the data space. The lower right quadrant of the 2-D data space shows the signals of target sequences Ill and IV detected individually in a partition. The upper right quadrant of the 2-D data space shows the signals of one KRAS and one BRAF target sequence (i.e. I+III, II+III, I+IV and II+IV) detected simultaneously in one partition. The signal intensity in the green detection channel is shown on the Y-axis and the signal intensity in the blue detection channel on the X-axis. Unit of the axes: relative fluorescence units (RFU).

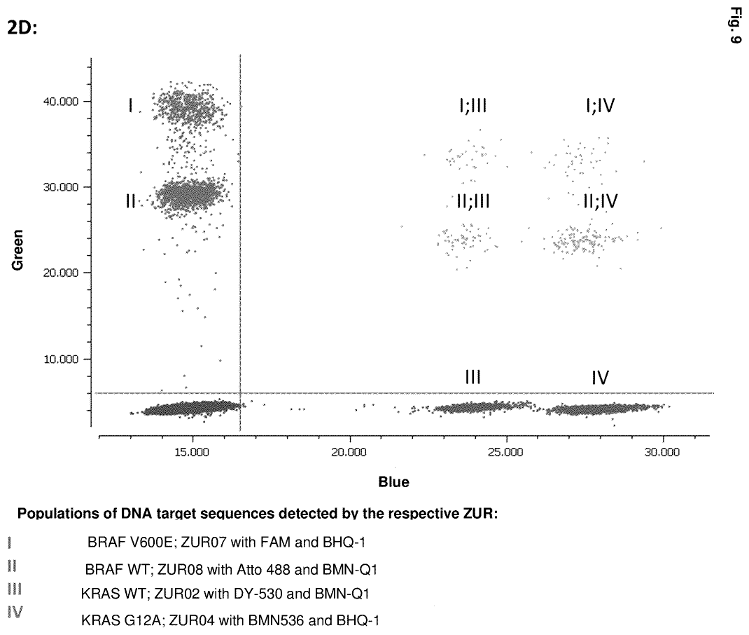

FIG. 10: The figure shows the results of example 6 in a three-dimensional (3-D) plot, wherein samples in the green, red and blue detection channels were analyzed for the presence of 6 target sequences simultaneously using a (direct) monochrome mediator probe multiplex PCR. For this purpose, the samples were amplified and analyzed in a corresponding digital PCR system (Stilla Naica system). In this “6-Plex Mediator Probe PCR”, specific target sequence-independent reporter molecules (ZUR02, ZUR04-ZUR08) were used for the following target sequences: I-BRAF V600E (BRAF gene mutation V600E; ZUR07, label: FAM/BHQ-1), II-BRAF WT (BRAF wild-type gene; ZUR08, label: Atto 488/BMN-W1), III-KRAS WT (KRAS wild-type gene; ZUR02, label: DY530/BMN-Q1), IV-KRAS G12A (KRAS gene mutation G12A; ZUR04, label: Cy5/BHQ-1), V-KRAS G12D (KRAS gene mutation G12D; ZUR05, label: Cy5/BHQ-2), VI-KRAS G12V (KRAS gene mutation G12V, ZUR06, label: Atto 647N/BHQ-2). The reporter molecules for BRAF target gene variants BRAF V600E and WT generated a signal in the blue detection channel (FAM or Atto 488 fluorophore) upon activation, the reporter molecules for KRAS WT and G12A target gene variants generated a signal in the green detection channel (DY-530 or BMN-536 fluorophore) and the reporter molecules for KRAS G12D and G12V target gene variants generated a signal in the red detection channel (Cy5 and Atto-647N fluorophore) upon activation. In the 3-D plot (3-D data space), the signals of target sequences I and II detected individually in a partition are shown on the X-axis. The signals of target sequences V and VI detected individually in a partition are shown on the Y-axis of the 3-D data space. The signals of target sequences Ill and IV detected individually in a partition are shown on the Z-axis of the 3-D data space. The signals of the respective KRAS or BRAF target sequences detected simultaneously in a partition are also shown in the 3-D data space. The signal intensity in the red detection channel is shown on the Y-axis, the signal intensity in the blue detection channel on the X-axis and the signal intensity in the green detection channel on the Z-axis. Unit of the axes: relative fluorescence units (RFU).

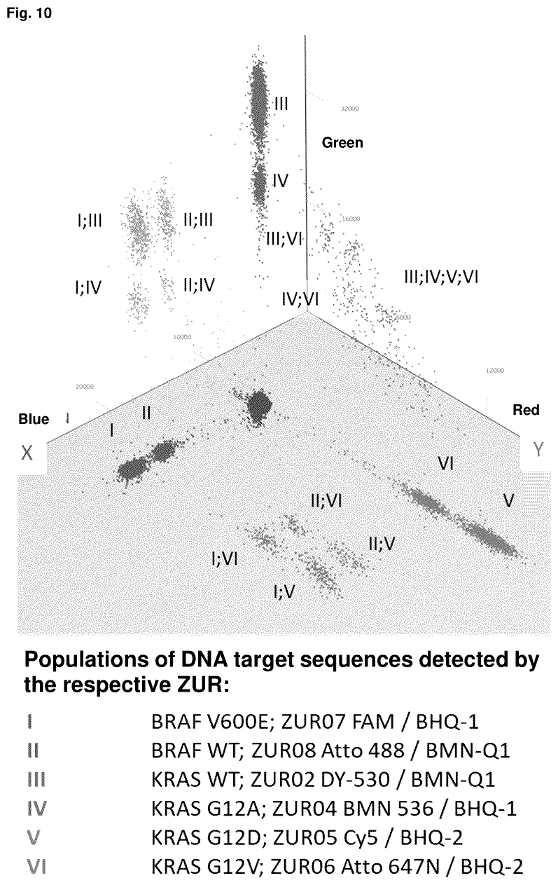

FIG. 11: One possibility for generating different signal strengths by using different labels on reporter molecules results in embodiments e.g. from the physical molecular properties of the labels, which are amplified by contact quenching after activation of the target sequence-unspecific reporter molecule. In both graphs, the X-axis shows the fixed dimensions of a label (or “marker”) and the measured signal, e.g. wavelength, potential, frequency, etc. The variable dimensions of the measured signal, in this case the signal strength, are shown on the Y-axis. The points in the respective graph show the intersection of the respective signal strengths 1 and 2 of fluorophore 1 or 2 and the respective measurement wavelength.

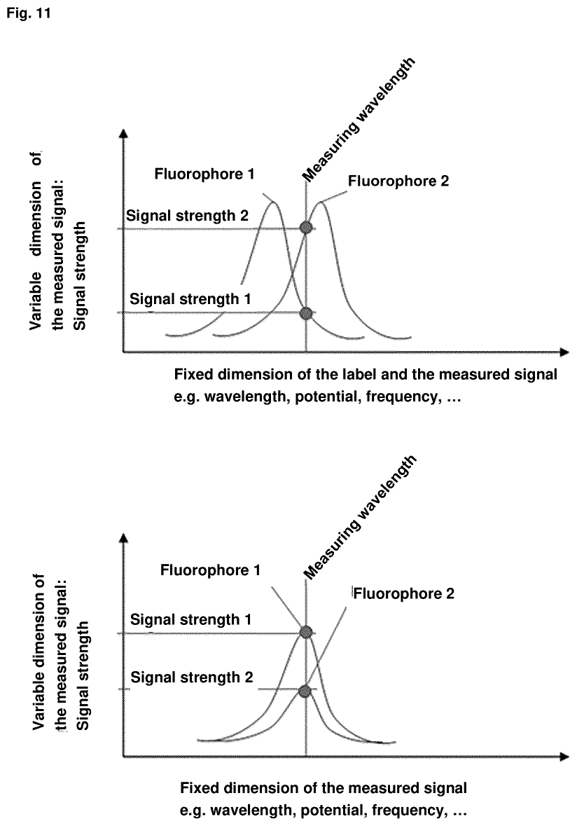

FIG. 12: Depiction of the results from example 9 in which three different DNA target sequences were detected in one detection channel by three different target sequence-unspecific reporter molecules, each with three different fluorescent labels, as part of a 3-plex mediator probe PCR with three different mediator probes in the infrared channel of the Prism6. In this “3-Plex Mediator Probe PCR”, the three target sequences I-KRAS G12D (KRAS single point mutation G12D), II KRAS WT (KRAS wild-type genome sequence) and III KRAS G12V (KRAS single point mutation G12V) were characterized by the three target sequence unspecific reporter molecules with the fluorophore quencher combinations Cy5.5+BHQ2 (I KRAS G12D), Dy636+BHQ2 (II KRAS WT) and Atto680+BHQ2 (III KRAS G12V). Each of these target sequence-unspecific reporter molecules thus generates a specific population in the data space, which can be assigned to one of the three DNA target sequences.

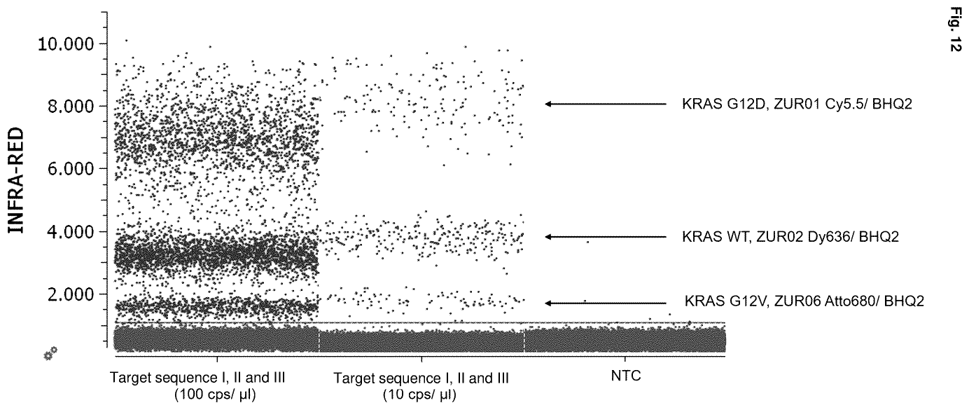

### BIBLIOGRAPHY

Faltin, Bernd; Wadle, Simon; Roth, Günter; Zengerle, Roland; Stetten, Felix von (2012): Mediator probe PCR: a novel approach for detection of real-time PCR based on label-free primary probes and standardized secondary universal fluorogenic reporters. In: Clinical chemistry 58 (11), pp. 1546-1556. DOI: 10.1373/clinchem.2012.186734.
Garcia-Murillas, Isaac; Schiavon, Gaia; Weigelt, Britta; Ng, Charlotte; Hrebien, Sarah; Cutts, Rosalind J. et al. (2015): Mutation tracking in circulating tumor DNA predicts relapse in early breast cancer. In: Science translational medicine 7 (302), 302ra133. DOI: 10.1126/scitranslmed.aab0021.
Hughesman, Curtis B.; Lu, X. J. David; Liu, Kelly Y. P.; Zhu, Yuqi; Poh, Catherine F.; Haynes, Charles (2016): A Robust Protocol for Using Multiplexed Droplet Digital PCR to Quantify Somatic Copy Number Alterations in Clinical Tissue Specimens. In: PloS one 11 (8), e0161274. DOI: 10.1371/journal.pone.0161274.
Kipf, Elena; Schlenker, Franziska; Borst, Nadine; Fillies, Marion; Kirschner-Schwabe, Renate; Zengerle, Roland et al. (2022): Advanced Minimal Residual Disease Monitoring for Acute Lymphoblastic Leukemia with Multiplex Mediator Probe PCR. In: The Journal of molecular diagnostics: JMD 24 (1), pp. 57-68. DOI: 10.1016/j.jmoldx.2021.10.001.
Lehnert, Michael; Kipf, Elena; Schlenker, Franziska; Borst, Nadine; Zengerle, Roland; Stetten, Felix von (2018): Fluorescence signal-to-noise optimisation for real-time PCR using universal reporter oligonucleotides. In: Anal. Methods 10 (28), pp. 3444-3454. DOI: 10.1039/C8AY00812D.
Milbury, Coren A.; Zhong, Qun; Lin, Jesse; Williams, Miguel; Olson, Jeff; Link, Darren R.; Hutchison, Brian (2014): Determining lower limits of detection of digital PCR assays for cancerrelated gene mutations. In: Biomolecular detection and quantification 1 (1), pp. 8-22. DOI: 10.1016/j.bdq.2014.08.001.
Pecoraro S., Berben G., Burns M., Corbisier P., De Giacomo M., De Loose M., Dagand E., Dobnik D., Eriksson R., Holst-Jensen A., Kagkli D. M., Kreysa J., Lievens A., Mäde D., Mazzara M., Paternò A., Peterseil V., Savini C., Sovová T., Sowa S., Spilsberg B.: Overview and recommendations for the application of digital PCR. JRC Technical Report. Luxembourg: Publications Office of the European Union, 2019 (2019). Available online at https://publications.jrc.ec.europa.eu/repository/handle/JRC115736.
Schlenker, Franziska; Kipf, Elena; Borst, Nadine; Hutzenlaub, Tobias; Zengerle, Roland; Stetten, Felix von; Juelg, Peter (2021a): Virtual Fluorescence Color Channels by Selective Photobleaching in Digital PCR Applied to the Quantification of KRAS Point Mutations. In: Analytical chemistry 93 (30), pp. 10538-10545. DOI: 10.1021/acs.analchem.1c01488.
Schlenker, Franziska; Kipf, Elena; Deuter, Max; Hoffkes, Inga; Lehnert, Michael; Zengerle, Roland et al. (2021b): Stringent Base Specific and Optimization-Free Multiplex Mediator Probe ddPCR for the Quantification of Point Mutations in Circulating Tumor DNA. In: Cancers 13 (22). DOI: 10.3390/cancers13225742.
Schuler, Friedrich; Trotter, Martin; Zengerle, Roland; Stetten, Felix von (2016): Monochrome Multiplexing in Polymerase Chain Reaction by Photobleaching of Fluorogenic Hydrolysis Probes. In: Analytical chemistry 88 (5), pp. 2590-2595. DOI: 10.1021/acs.analchem.5b02960.
Whale, Alexandra S.; Huggett, Jim F.; Tzonev, Svilen (2016): Fundamentals of multiplexing with digital PCR. In: Biomolecular detection and quantification 10, pp. 15-23. DOI: 10.1016/j.bdq.2016.05.002.
Corné, Julien; Le Du, Fanny; Quillien, Véronique; Godey, Florence; Robert, Lucie; Bourien, H\tilde{e}loïse et al. (2021): Development of multiplex digital PCR assays for the detection of PIK3CA mutations in the plasma of metastatic breast cancer patients. In: Scientific reports 11 (1), p. 17316. DOI: 10.1038/s41598-021-96644-6.
Faltin, Bernd; Wadle, Simon; Roth, Günter; Zengerle, Roland; Stetten, Felix von (2012): Mediator probe PCR: a novel approach for detection of real-time PCR based on label-free primary probes and standardized secondary universal fluorogenic reporters. In: Clinical chemistry 58 (11), pp. 1546-1556. DOI: 10.1373/clinchem.2012.186734.
Garcia-Murillas, Isaac; Schiavon, Gaia; Weigelt, Britta; Ng, Charlotte; Hrebien, Sarah; Cutts, Rosalind J. et al. (2015): Mutation tracking in circulating tumor DNA predicts relapse in early breast cancer. In: Science translational medicine 7 (302), 302ra133. DOI: 10.1126/scitranslmed.aab0021.
Hughesman, Curtis B.; Lu, X. J. David; Liu, Kelly Y. P.; Zhu, Yuqi; Poh, Catherine F.; Haynes, Charles (2016): A Robust Protocol for Using Multiplexed Droplet Digital PCR to Quantify Somatic Copy Number Alterations in Clinical Tissue Specimens. In: PloS one 11 (8), e0161274. DOI: 10.1371/journal.pone.0161274.
Kipf, Elena; Schlenker, Franziska; Borst, Nadine; Fillies, Marion; Kirschner-Schwabe, Renate; Zengerle, Roland et al. (2022): Advanced Minimal Residual Disease Monitoring for Acute Lymphoblastic Leukemia with Multiplex Mediator Probe PCR. In: The Journal of molecular diagnostics: JMD 24 (1), pp. 57-68. DOI: 10.1016/j.jmoldx.2021.10.001.
Lehnert, Michael; Kipf, Elena; Schlenker, Franziska; Borst, Nadine; Zengerle, Roland; Stetten, Felix von (2018): Fluorescence signal-to-noise optimisation for real-time PCR using universal reporter oligonucleotides. In: Anal. Methods 10 (28), pp. 3444-3454. DOI: 10.1039/C8AY00812D.
Madic, Jordan; Jovelet, Cécile; Lopez, Julien; André, Barbara; Fatien, Jean; Miran, Isabelle et al. (2018): EGFR C797S, EGFR T790M and EGFR sensitizing mutations in non-small cell lung cancer revealed by six-color crystal digital PCR. In: Oncotarget 9 (100), pp. 37393-37406. DOI: 10.18632/oncotarget.26446.
Milbury, Coren A.; Zhong, Qun; Lin, Jesse; Williams, Miguel; Olson, Jeff; Link, Darren R.; Hutchison, Brian (2014): Determining lower limits of detection of digital PCR assays for cancerrelated gene mutations. In: Biomolecular detection and quantification 1 (1), pp. 8-22. DOI: 10.1016/j.bdq.2014.08.001.
Pecoraro S., Berben G., Burns M., Corbisier P., De Giacomo M., De Loose M., Dagand E., Dobnik D., Eriksson R., Holst-Jensen A., Kagkli D. M., Kreysa J., Lievens A., Made D., Mazzara M., Paternò A., Peterseil V., Savini C., Sovová T., Sowa S., Spilsberg B.: Overview and recommendations for the application of digital PCR. JRC Technical Report. Luxembourg: Publications Office of the European Union, 2019 (2019). Available online at https://publications.jrc.ec.europa.eu/repository/handle/JRC115736.
Schlenker, Franziska; Kipf, Elena; Borst, Nadine; Hutzenlaub, Tobias; Zengerle, Roland; Stetten, Felix von; Juelg, Peter (2021a): Virtual Fluorescence Color Channels by Selective Photobleaching in Digital PCR Applied to the Quantification of KRAS Point Mutations. In: Analytical chemistry 93 (30), pp. 10538-10545. DOI: 10.1021/acs.analchem.1c01488.
Schlenker, Franziska; Kipf, Elena; Deuter, Max; Höffkes, Inga; Lehnert, Michael; Zengerle, Roland et al. (2021b): Stringent Base Specific and Optimization-Free Multiplex Mediator Probe ddPCR for the Quantification of Point Mutations in Circulating Tumor DNA. In: Cancers 13 (22). DOI: 10.3390/cancers13225742.
Schuler, Friedrich; Trotter, Martin; Zengerle, Roland; Stetten, Felix von (2016): Monochrome Multiplexing in Polymerase Chain Reaction by Photobleaching of Fluorogenic Hydrolysis Probes. In: Analytical chemistry 88 (5), pp. 2590-2595. DOI: 10.1021/acs.analchem.5b02960.
Stilla Technologies: High multiplex, ultrasensitive EGFR detection using the EGFR 6-color Crystal Digital PCR™ kit. Stilla Technologies. Available online at https://www.stillatechnologies.com/wpcontent/uploads/2021/12/StillaTechnologies-6-color-EGFR-kit-App-Note.pdf.
Whale, Alexandra S.; Huggett, Jim F.; Tzonev, Svilen (2016): Fundamentals of multiplexing with digital PCR. In: Biomolecular detection and quantification 10, pp. 15-23. DOI: 10.1016/j.bdq.2016.05.002.

1. A method for the detection of at least two nucleic acid target sequences by at least two target sequence-independent reporter molecules in the same detection channel, comprising:
a) providing at least a first and a second nucleic acid target sequence,
b) providing at least a first and a second mediator probe, each comprising an oligonucleotide,
wherein the oligonucleotide of the first mediator probe comprises a mediator sequence, and a probe sequence, wherein the mediator sequence has an affinity for a target sequence-independent reporter molecule of a first type and the probe sequence exhibits an affinity for the first nucleic acid target sequence,
wherein the oligonucleotide of the second mediator probe comprises a mediator sequence and a probe sequence, wherein the mediator sequence has an affinity for a target sequence-independent reporter molecule of a second type and the probe sequence exhibits an affinity for the second nucleic acid target sequence,
wherein the at least first and second mediator probes have no signal-generating labels,
c) providing the at least two target sequence-independent reporter molecules, each comprising
at least one label with a measurable signal in the same detection channel, and
a nucleic acid sequence which has a specific affinity for at least one of the mediator sequences,
wherein the at least one label of each type of the at least two target sequence-independent reporter molecules provides a signal intensity which is distinguishable from the signal intensities of the labels of all other target sequence-independent reporter molecule types and enables direct assignment to the respective nucleic acid target sequence,
d) performing a nucleic acid detection reaction, wherein the at least one mediator sequence of at least a first mediator probe is released when its probe sequence binds to the at least a first nucleic acid target sequence resulting in a released mediator sequence,
wherein the at least one released mediator sequence binds to the at least one target sequence-independent reporter molecule of a first type resulting in a bound mediator sequence, wherein the at least one label generates a signal which, due to its signal intensity and/or emission spectrum, is characteristic of the first nucleic acid target sequence assigned to the respective target sequence-independent reporter, to which the at least one probe sequence of the at least one first mediator probe has bound,
e) detecting the signal generated in d), comprising the detection of the signal in the detection channel and/or an analysis of the signal, a signal strength and/or an emission spectrum of the signal.

2. The method according to claim 1, wherein the at least one label of a target sequence-independent reporter molecule is at least one fluorophore and/or at least one quencher.

3. The method according to claim 1, wherein the analysis of the signal, the signal strength and/or the emission spectrum of the signal in e) comprises representation and/or the analysis of the signal detected depending on the signal strength and/or the detection channel and/or the emission spectrum in a data space spanned by the evaluated detection channels.

4. The method according to claim 1, wherein the different target sequence independent reporter molecule types differ in the signal intensity and/or the emission spectrum of their at least one label.

5. The method according to claim 1, wherein the at least one label of a target sequence-independent reporter molecule comprises at least two fluorophores and/or two quenchers having the same or different emission spectrum and/or the same or different signal intensity.

6. The method according to claim 1, wherein the at least one label of a target sequence-independent reporter molecule comprises at least one fluorophore and at least one quencher,
wherein contact quenching occurs between the at least one fluorophore and the at least one quencher as long as none of the mediator sequences binds to the corresponding target sequence-independent reporter molecule, or as long as a bound mediator sequence has not been extended during the nucleic acid detection reaction.

7. The method according to claim 1, wherein the label of the at least one target sequence-independent reporter molecule comprises at least two complementary opposing nucleobases each having at least one label or at least two opposing bases offset by one base position from a complementary base pairing each having at least one label.

8. The method according to claim 1, wherein in d) to e) n different nucleic acid target sequences are indirectly detected by n different target sequence-independent reporter molecule types, wherein the detection of the signal generated in d) takes place in k detection channels,
where n>k and n≥2, and
wherein at least two different target sequence-independent reporter molecule types are detected in the same detection channel in e) and/or represented in the same region of a data space in the representation under e).

9. The method according to claim 1, wherein the signal of the label of a target sequence-independent reporter molecule is generated by cleavage and/or separation of the target sequence-independent reporter molecule and/or by spatial separation of at least one fluorophore and at least one quencher, when the at least one label of a target sequence-independent reporter molecule is the at least one fluorophore and/or the at least one quencher.

10. The method according to claim 1, wherein one of the at least two target sequence-independent reporter molecule is an oligonucleotide or an oligonucleotide complex.

11. The method according to claim 1, wherein the nucleic acid detection reaction in d) comprises an amplification method for DNA and/or cDNA.

12. The method according to claim 11, wherein the amplification in d) is a PCR, RT-PCR, RPA or LAMP, and wherein in the course of DNA amplification the mediator sequence of a one of the mediator probes bound to one of the nucleic acid target sequences is released by an enzymatic activity of a biomolecule, wherein said mediator probe then binds to one of the target sequence-independent reporter molecules such that the signal is generated.

13. The method according to claim 11, wherein the detection in e) is carried out in context of a digital amplification and/or signal generation.

14. The method according to claim 1, wherein the target sequence-independent reporter molecules are universal reporters and/or modular reporter complexes and the at least one released mediator sequence is a component of a Mediator Probe PCR or Mediator Displacement LAMP.

15. The method according to claim 1, wherein the at least one nucleic acid target sequence resulted from a conversion of another biomolecule into DNA sequence information.

16. A kit for carrying out the method according to claim 1, comprising:
the at least one target sequence-independent reporter molecule of a first type comprising at least one label,
the at least one target sequence-independent reporter molecule of a second type comprising at least one label,
the at least one first mediator probe whose mediator sequence has the affinity for the at least one target sequence-independent reporter molecule of the first type, and whose oligonucleotide sequence exhibits the affinity for the first nucleic acid target sequence,
the at least one second mediator probe whose mediator sequence has the affinity for the at least one target sequence-independent reporter molecule of the second type, and whose oligonucleotide sequence exhibits the affinity for the second nucleic acid target sequence,
optionally at least one buffer,
optionally a polymerase,
optionally a reverse transcriptase, and
optionally at least one PCR primer.


SEQUENCE LISTING (26 sequences)

SEQ ID NO: 1 - DNA, 18 bp, synthetic construct
  source 1..18: mol_type = other DNA
ggcctgctgaaaatgact

SEQ ID NO: 2 - DNA, 25 bp, synthetic construct
  source 1..25: mol_type = other DNA
acaaaatgattctgaattagctgta

SEQ ID NO: 3 - DNA, 21 bp, synthetic construct
  source 1..21: mol_type = other DNA
gacccactccatcgagatttc

SEQ ID NO: 4 - DNA, 23 bp, synthetic construct
  source 1..23: mol_type = other DNA
gcttgctctgataggaaaatgag

SEQ ID NO: 5 - DNA, 43 bp, synthetic construct
  source 1..43: mol_type = other DNA
ctccagttcggtccagctccaactaccacaagtttatattcag

SEQ ID NO: 6 - DNA, 39 bp, synthetic construct
  source 1..39: mol_type = other DNA
gatacagggtccactggcgtaggcaagagtgccttgacg

SEQ ID NO: 7 - DNA, 41 bp, synthetic construct
  source 1..41: mol_type = other DNA
atgtcccaggtgcatggcgtaggcaagagtgccttgacgat

SEQ ID NO: 8 - DNA, 40 bp, synthetic construct
  source 1..40: mol_type = other DNA
aggtaggctcacttggcgtaggcaagagtgccttgacgat

SEQ ID NO: 9 - DNA, 54 bp, synthetic construct
  source 1..54: mol_type = other DNA
acatgctcatgttgtgtctgtagctagaccaaaatcacctatttttactgtgag

SEQ ID NO: 10 - DNA, 54 bp, synthetic construct
  source 1..54: mol_type = other DNA
gtgttcctcacatgctactgtagctagaccaaaatcacctatttttactgtgag

SEQ ID NO: 11 - DNA, 45 bp, synthetic construct
  source 1..45: mol_type = other DNA
  modified_base 1: mod_base = OTHER; note = BMN-Q1
  modified_base 22: mod_base = OTHER; note = DY-530
gaccggccaagacgcgccggtctgttcactgaccgaactggagca

SEQ ID NO: 12 - DNA, 45 bp, synthetic construct
  source 1..45: mol_type = other DNA
  modified_base 1: mod_base = OTHER; note = BHQ-1
  modified_base 22: mod_base = OTHER; note = BMN-536
gaccgcactagtagatgcggtctgtcgtggaccctgtatcgagca

SEQ ID NO: 13 - DNA, 46 bp, synthetic construct
  source 1..46: mol_type = other DNA
  modified_base 1: mod_base = OTHER; note = BHQ-2
  modified_base 22: mod_base = OTHER; note = Cy5
gaccggctaagacgcgccggtctgttgcacctgggacatcgactat

SEQ ID NO: 14 - DNA, 46 bp, synthetic construct
  source 1..46: mol_type = other DNA
  modified_base 1: mod_base = OTHER; note = BHQ-2
  modified_base 22: mod_base = OTHER; note = Atto-647N
gacgcgtagtacagaacgcgtctgttcagtgagcctacctgccttc

SEQ ID NO: 15 - DNA, 47 bp, synthetic construct
  source 1..47: mol_type = other DNA
  modified_base 1: mod_base = OTHER; note = BHQ-1
  modified_base 20: mod_base = OTHER; note = FAM
atgccgattagatgcggcattcgatcacacaacatgagcatgtgtac

SEQ ID NO: 16 - DNA, 45 bp, synthetic construct
  source 1..45: mol_type = other DNA
  modified_base 1: mod_base = OTHER; note = BMN-Q1
  modified_base 20: mod_base = OTHER; note = Atto-488
atccgccaagacgcgcggattagcatgtgaggaacacgatgacac

SEQ ID NO: 17 - DNA, 160 bp, unidentified
  source 1..160: mol_type = other DNA
caaactgatgggacccactccatcgagatttcactgtagctagaccaaaatcacctatttttactgtgaggtcttcatgaagaaatatatctgaggtgtagtaagtaaaggaaaacagtagatctcattttcctatcagagcaagcattatgaagagttt

SEQ ID NO: 18 - DNA, 160 bp, unidentified
  source 1..160: mol_type = other DNA
caaactgatgggacccactccatcgagatttctctgtagctagaccaaaatcacctatttttactgtgaggtcttcatgaagaaatatatctgaggtgtagtaagtaaaggaaaacagtagatctcattttcctatcagagcaagcattatgaagagttt

SEQ ID NO: 19 - DNA, 131 bp, unidentified
  source 1..131: mol_type = other DNA
cattttcattatttttattataaggcctgctgaaaatgactgaatataaacttgtggtagttggagctggtggcgtaggcaagagtgccttgacgatacagctaattcagaatcattttgtggacgaatat

SEQ ID NO: 20 - DNA, 131 bp, unidentified
  source 1..131: mol_type = other DNA
cattttcattatttttattataaggcctgctgaaaatgactgaatataaacttgtggtagttggagctgctggcgtaggcaagagtgccttgacgatacagctaattcagaatcattttgtggacgaatat

SEQ ID NO: 21 - DNA, 131 bp, unidentified
  source 1..131: mol_type = other DNA
cattttcattatttttattataaggcctgctgaaaatgactgaatataaacttgtggtagttggagctgatggcgtaggcaagagtgccttgacgatacagctaattcagaatcattttgtggacgaatat

SEQ ID NO: 22 - DNA, 131 bp, unidentified
  source 1..131: mol_type = other DNA
cattttcattatttttattataaggcctgctgaaaatgactgaatataaacttgtggtagttggagctgttggcgtaggcaagagtgccttgacgatacagctaattcagaatcattttgtggacgaatat

SEQ ID NO: 23 - DNA, 43 bp, synthetic construct
  source 1..43: mol_type = other DNA
ggctctacgaccaacatggcgtaggcaagagtgccttgacgat

SEQ ID NO: 24 - DNA, 46 bp, synthetic construct
  source 1..46: mol_type = other DNA
  modified_base 1: mod_base = OTHER; note = BHQ-2
  modified_base 22: mod_base = OTHER; note = Cy5.5
gaccggccaagacgcgccggtctgttggtcgtagagcccagaacga

SEQ ID NO: 25 - DNA, 45 bp, synthetic construct; identical to SEQ ID NO: 11
  source 1..45: mol_type = other DNA
  modified_base 22: mod_base = OTHER; note = Dy636
  modified_base 1: mod_base = OTHER; note = BHQ-2

SEQ ID NO: 26 - DNA, 46 bp, synthetic construct; identical to SEQ ID NO: 14
  source 1..46: mol_type = other DNA
  modified_base 22: mod_base = OTHER; note = Atto680
  modified_base 1: mod_base = OTHER; note = BHQ-2